(19) United States
(12) Patent Application Publication
(10) Pub. No.: US 2009/0186077 A1
(43) Pub. Date: Jul. 23, 2009

(54) THERMO-RESPONSIVE MATERIALS

(76) Inventors: Jackie Y. Ying, Wincherster, MA (US); Cindy Ren, Arcadia, CA (US)

Correspondence Address: Pearl Cohen Zedek Latzer, LLP, 1500 Broadway, 12th Floor, New York, NY, 10036, US

(21) Appl. No.: 12/279,461

(22) PCT Filed: Feb. 14, 2007
(86) PCT No.: PCT/US07/03679

§ 371 Date: Dec. 10, 2008

Related U.S. Application Data
(60) Provisional application No. 60/773,318, filed on Feb. 15, 2006.
(60) Provisional application No. 60/775,325, filed on Feb. 22, 2006.

(51) Int. Cl. A61K 9/127; A61K 35/00

(52) U.S. Cl. 424/450; 424/93.1

(57) ABSTRACT

The invention is directed to, inter alia, injectable materials, which comprise a a zero-length cross-linking agent encapsulated in a thermo-responsive liposome dispersed in a polymer solution where the polymer and cross-linking agent are segregated at a first temperature, and non-segregated at a second temperature, and the material is injectable at the first temperature. The invention is directed to, inter alia, processes for producing the material and use of the material for tissue repair and regeneration and controlled delivery of an agent are also described.

### FIELD OF INVENTION

This invention relates to matrices, processes of producing the same and their use in biological applications, such as in tissue engineering and/or repair or controlled delivery of an agent of interest.

### BACKGROUND OF THE INVENTION

Current methods for treating organ and tissue damage include transplantation, reconstruction, and implantation of prosthetics, however, these solutions are associated with problems of donor shortage, immunological rejection, and inadequate replacement of biological function. In recent years, tissue engineering has emerged as an alternative therapy. Tissue engineering involves the construction of an implantable biological device that either elicits tissue regeneration or replaces damaged tissue. This is generally accomplished by three components: (1) patient or donor cells, (2) regulators that guide the regenerative activity of the cells, and (3) a scaffold that acts as a delivery vehicle for the cells and regulators, and as a substrate for tissue growth. By introducing cells on a matrix conducive to the synthesis of new tissue, the body's natural regenerative ability is enhanced. Several studies have already shown the feasibility of tissue engineering in various organs, such as liver, skin, bone, and cartilage.

Significant research has been devoted to the development of suitable scaffolds, as it is a key component in tissue engineering. It must act as an extracellular matrix (ECM), provide mechanical support, and guide cell growth. Currently, several limitations remain in the development of an appropriate scaffold. First, implantation of the scaffold requires invasive surgery, which inherently involves risks, such as infection and failure to heal. Secondly, it is difficult to mold a material to perfectly fit a defect site. A poorly fitting scaffold can result in failure of the newly formed tissue to integrate with the host tissue. This may subsequently lead to dead space and implant instability. Dead space can cause fluid accumulation and chronic inflammation, while implant instability can cause micromotion, producing unfavorable forces at the construct interface. Both dead space and implant instability would result in the degradation and limited lifetime of the reparative tissue.

In order to address these issues, injectable scaffolds have been investigated as a possible approach to tissue engineering. This method is based on the injection of a liquid that cures at the defect site. The liquid is typically composed of a soluble monomer or polymer that forms a hydrogel by polymerization, phase transition or crosslinking. This approach not only eliminates the need for surgery, but it also allows scaffold formation to occur in situ, thereby allowing the scaffold to take the shape of the defect. This would minimize dead space as well as improve the integration of the scaffold with the existing tissue, due to the intimate contact of the polymer with the surrounding tissue during scaffold formation. and the mechanical interlocking that occurs due to surface microroughness.

Two important properties to consider in an injectable scaffold are workability and mechanical integrity. The working time of the scaffold must be long enough for the clinician to handle and inject it, while the strength of the final scaffold must be sufficient to withstand physiological stresses, particularly in applications such as bone and cartilage. In a typical polymer-crosslinker system, it is common to control both characteristics by varying the crosslink density, but the two requirements often conflict with each other since the mechanical strength tends to decrease as curing time increases. In order to make a scaffold practical to work with, the strength and, consequently, the efficacy of the scaffold is often compromised.

### SUMMARY OF THE INVENTION

In one embodiment, this invention provides an injectable scaffolding material, comprising:
a. a polymer solution;
b. a thermno-responsive liposome dispersed in said solution; and
c. a zero-length cross-linking agent encapsulated in said liposome;
   wherein said polymer and said cross-linking agent are segregated at a first temperature, non-segregated at a second temperature, and said scaffolding material is injectable at said first temperature.

According to this aspect, and in one embodiment, the polymer solution comprises poly (pyranose), poly(hydroxyl acid), poly(lactone), poly (amino acid), poly(anhydride), poly (orthoester), poly (phosphazine), poly(ethylene glycol) or poly(phosphoester). In another embodiment, the polymer solution comprises a collagen, a glycosaminoglycan, or a combination thereof. In one embodiment, the concentration of the polymer ranges from 0.1-50% w/w, or in another embodiment, 45-95% w/w of the material.

In one embodiment, the thermo-responsive liposome comprises a mixture of lipids, phospholipids, or a combination thereof. In one embodiment, the lipid is cholesterol. In another embodiment, the phospholipid is a dipalmitoyl-phosphatidylcholine (DPPC), dimyristoylphosphatidylcholine (DMPC), distearoyl-phosphatidylcholine or a combination thereof. In one embodiment, the liposome comprises two or more phospholipids. In another embodiment, the relative concentration of one of said two or more phospholipids is varied, so as to increase permeability of said liposome. In another embodiment, the liposome further comprises a lipid, which inreases fluidity of said liposome.

In one embodiment, the first temperature is less than the phase transition temperature of the mixture. In another embodiment, the second temperature is about 37° C.

In one embodiment, the zero-length cross-linking agent is (1 ethyl 3-(3dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or a combination thereof. In another embodiment, the zero-length cross-linking agent is an enzyme, which in one embodiment, is a transglutamninase, peroxidase, xanthine oxidase, or a combination thereof.

In one embodiment, the scaffolding material comprises a compound or cell of interest.

In another embodiment, the invention provides a cosmetic filler, drug delivery vehicle or scaffold for tissue engineering or repair, comprising the injectable scaffolding material as herein described.

In another embodiment, this invention provides a process for preparing an injectable scaffolding material, comprising the steps of:
a. preparing a mixture of lipids, phopsholipids, or a combination thereof and a cross-linking agent;
b. freezeing said mixture;
c. rehydrating said mixture to form liposomes encapsulating said cross-linking agent; and
d. dispersing said liposomes in a liquid comprising a polymer
   wherein said dispersing is conducted at a temperature which is less than the phase transition temperature of said mixture and said cross-linking agent or reactive species and said polymer are segregated at said temperature.

In one embodiment, the freezing is accomplished at a rate of between 0.5-100° C. per minute. In another embodiment, the method further comprises the step of washing off encapsulated cross linking agent. In another embodiment, the rehydrating is conducted with a salt solution comprising a monovalent or divalent cation. In one embodiment, the salt solution comprises sodium chloride.

In one embodiment, the method further comprises the step of sonicating said mixture prior to freezing, in step (b).

In another embodiment, the rehydrating is accomplished in a small volume, which in some embodiments ranges from about 1/10 to about 1/1 of the volume of the liposome solution.

In some embodiments, this invention provides an injectable scaffolding material prepared according to a process as herein described.

In another embodiment, this invention provides a method or tissue repair, regeneration, or organogenesis in a subject, the method comprising:
a. administering a scaffolding material to a subject, said scaffolding material comprising:
   ∘ a polymer solution;
   ∘ a thermo-responsive liposome dispersed in said solution; and
   ∘ a zero-length cross-linking agent encapsulated in said liposome;
      wherein said polymer and said cross-linking agent are segregated at a first temperature, non-segregated at a second temperature, and said scaffolding material is injectable at said first temperature.

According to this aspect of the invention, and in one embodiment, the scaffolding material further comprises cells involved in tissue repair, regeneration, or organogenesis. In another embodiment, the scaffolding material further comprises a tissue promoting factor, which in one embodiment, is a hormone, a nucleic acid, a growth factor, a chemokine, a peptide, an enzyme or a combination thereof.

In some embodiments, the tissue repair or regeneration is of the skin. In some embodiments, the repair or regeneration is of a skin wrinkle or lesion, and in some embodiments, the injectable scaffolding material serves as a filler for a skin wrinkle or lesion in the subject.

In some embodiments, this invention provides a method of controlled delivery of an agent in a subject, the method comprising administering a controlled delivery material to a subject, the material comprising:

∘ a polymer solution;

∘ a thermo-responsive liposome dispersed in the solution;

∘ a zero-length cross-linking agent encapsulated in the liposome; and

∘ at least one agent of interest encapsulated in the liposome;

wherein the polymer and the cross-linking agent are segregated at a first temperature, non-segregated at a second temperature, and the controlled delivery material is injectable at the first temperature.

In some embodiments, the controlled delivery material comprises two or more agents. In some embodiments, the second agent of interest is dispersed in the polymer solution. In some embodiments, the second agent is a precursor molecule, and the at least one agent processes the precusor to form a final product for delivery to the subject.

### BRIEF DESCRIPTION OF THE DRAWINGS

FIG. 1A depicts thermoresponsive liposomes containing the cross-linker dispersed in a polymer solution, remaining as a liquid at room temperature. Upon exposure to body temperature, the cross-linker is released and reacts with the polymer to form a hydrogel network.

FIG. 1B depicts the crosslinking reaction of HA-Tyr in the presence of HRP and H₂O₂.

FIG. 2 plots thermally triggered HRP release from liposomes at (▪) 25° C. and (∘) 37° C.

FIG. 3 plots the effect of DPPC/DMPC weight ratio on HRP release from liposomes: (⋄,▴) 14/6, (∘,▪) 15/5 and (*,▪) 16/4 at (⋄,∘,*) 37° C. and (▴,▪,) 25° C.

FIG. 4 plots the effect of cholesterol on the (▪) total HRP release from liposomes after one hour at 37° C. and (⋄) liposome size.

FIG. 5 plots the effect of lipid concentration (A) and freezing method (B) on the total BRP release after 1 hour at 37° C.

FIG. 6 plots the effect of trehalose concentration, (_{▪}) 0 wt % or (_{▪}) 4 wt % on total HRP release after 1 hour at 25° C. and 37° C., respectively.

FIG. 7 depicts the effect of rehydration volume on total HRP release following 1 hour at 37° C.

FIG. 8 demonstrates the effect of sonication on the (□) total HRP release following 1 hour at 37° C. and (⋄) liposome size.

FIG. 9 plots the effect of HRP concentration on total HRP release after 1 hour at 37° C.

FIG. 10 demonstrates the G′ of HA-Tyr/H₂O₂/HRP liposome solution at ( - - - (lower line)) 20° C. and ( - - - (upper line)) 37° C., respectively.

FIG. 11 plots the elastic modulus versus gel point of (▴) HA-Tyr/H₂O₂/HRP solution and (▪) HA-Tyr/H₂O₂/HRP liposomes.

FIG. 12 demonstrates the effect of liposome wash solutions on the gelation point of HA-Tyr/H₂O₂/HRP liposome mixture at (^{▪}) 20° C. and (^{▪}) 37° C.

FIG. 13 is a micrograph depicting an intact gel harvested 2 hours after injection.

FIG. 14 are micrographs probed with Safranin ◯ staining for GAG content at a) Day 14 and b) Day 28, and Masson's trichrome staining for collagen content at c) Day 14 and d) Day 28.

FIG. 15 plots thrombin release from liposomes at (⋄)25° C. and (▪)37° C.

### DETAILED DESCRIPTION OF THE INVENTION

The invention is directed to, in some embodiments, an injectable scaffolding material, in-situ formed, solid scaffolding material, and their use in biological applications, such as in the preparation of tissue adhesives, tissue enginnering and/or repair.

In some embodiments, this invention provides a versatile platform for creating a thermally triggered injectable scaffold for tissue engineering.

In some embodiments, scaffold material preparation involves the encapsulation of a cross-linker, or active species, within thermoresponsive liposomes that are then dispersed within a polymer solution. At room temperature, the polymer and cross-linker or active species are segregated from each other, and therefore, the system remains a liquid that can readily be introduced, via for example, a syringe. In some embodiments, following injection and exposure to body temperature (37° C.), the cross-linker is released and scaffold solidification occurs.

This invention provides, in one embodiment, an injectable scaffolding material, comprising:
a. a polymer solution;
b. a thermo-responsive liposome dispersed inr said solution; and
c. a zero-length cross-linking agent encapsulated in said liposome;
   wherein said polymer and said cross-linking agent are segregated at a first temperature, non-segregated at a second temperature, and said scaffolding material is injectable at said first temperature.

According to this aspect, and in one embodiment, the polymer solution comprises poly (pyranose), poly(hydroxyl acid), poly(lactone), poly (amino acid), poly(anhydride), poly (orthoester), poly (phosphazine), poly(ethylene glycol) or poly(phosphoester). In another embodiment, the polymer solution comprises a polysaccharide, a collagen, a glycosaminoglycan, or a combination thereof. In one embodiment, the concentration of the polymer ranges from 1-85% w/w of the material. In one embodiment, the concentration of the polymer ranges from 0.1-50% w/w of the material, or in another embodiment, the concentration of the polymer ranges from 45-95% w/w of the material, or in another embodiment, the concentration of the polymer ranges from 0.1-30% w/w of the material, , or in another embodiment, the concentration of the polymer ranges from 25-75% w/w of the material, or in another embodiment, the concentration of the polymer ranges from 30-80% w/w of the material, or in another embodiment, the concentration of the polymer ranges from 15-45% w/w of the material.

In one embodiment, the polymer solution comprises a synthetic or natural polymer, such as a poly(cianoacrylate), poly(alkyl-cianoacrylate), poly(ketal), poly(caprolactone), poly(ε-caprolactone), poly(acetal), poly(α-hydroxy-ester), poly(β-hydroxy-ester), poly(hydroxyl-alkanoate), poly(propylene-fumarate), poly (imino-carbonate), poly(ester), poly(ethers), poly(carbonates), poly(amide), poly(urethane), poly(siloxane), poly(silane), poly(sulfide), poly(imides), poly(urea), poly(amide-enamine), polysaccharide, poly(organic acid), poly(electrolytes), poly(p-dioxanone), poly(olefin), poloxamer, inorganic or organomatallic polymers, elastomer, poly(saccharide), poly(organic acid), or any of their derivatives, or a copolymer obtained by a combination thereof. In one embodiment, the polymer solution comprises poly(D,L-lactide-co-glycolide) (PLGA). In another embodiment, the polymer solution comprises poly(D,L-lactide) (PLA). In another embodiment, the polymer solution comprises poly(D,L- glycolide) (PGA).

In one embodiment, the polymer solution may comprise proteins such as zein, modified zein, casein, gelatin, gluten, serum albumin, collagen, actin, α-fetoprotein, globulin, macroglobulin, cohesin, laminin, fibronectin, fibrinogen, osteocalcin, osteopontin, osteoprotegerin, or others, as will be appreciated by one skilled in the art. In another embodiment, the polymer solution may comprise cyclic sugars, cyclodextrins, synthetic derivatives of cyclodextrins, glycolipids, glycosaminoglycans, oligosaccharide, polysaccharides such as alginate, carrageenan (χ, λ, μ, κ), chitosane, celluloses, condroitin sulfate, curdlan, dextrans, elsinan, furcellran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluron acid and polyhyaluronic acid, chitin, poly(3-hydroxyalkanoate)s, such as poly(β-hydroxybutyrate), poly(3-hydroxyoctanoate) or poly(3-hydroxyfatty acids). In another embodiment, the polymer solution may comprise chemical derivatives thereof (substitutions, additions of chemical groups, for example, alkyl, alkylene, hydroxylations, oxidations, and other modifications routinely made by those skilled in the art), blends of, e.g. proteins or carbohydrates alone or in combination with synthetic polymers.

In one embodiment, the polymer solution comprises synthetically modified natural polymers, and may include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

In one embodiment, the polymer solution comprises synthetic degradable polymers, which may include, but are not limited to polyhydroxy acids, such as polylactides, polyglycolides and copolymers thereof; poly(ethylene terephthalate); poly(hydroxybutyric acid); poly(hydroxyvaleric acid); poly[lactide-co-(ε-caprolactone)]; poly[glycolide-co(ε-caprolactone)]; polycarbonates, poly(pseudo amino acids); poly(amino acids); poly(hydroxyalkanoate)s; polyanhydrides; polyortho esters; and blends and copolymers thereof.

In one embodiment, the polymer solution comprises a bioerodible polymer such as poly(lactide-co-glycolide)s, polyanhydrides, and polyorthoesters, which have carboxylic groups exposed on the external surface as the smooth surface of the polymer erodes, which may also be used. In one embodiment, the polymer contains labile bonds, such as polyanhydrides and polyesters.

In some embodiments, the polymer solutions will comprise agents of interest, which when implanted in a subject provide a desired effect. In some embodiments, the desired effect is manifested upon raising of the temperature of the implanted material, as a function of exposure to a particular temperature. In some embodiments, the desired effect is manifested upon raising of the temperature of the implanted material, as a function of exposure to a component of the liposome contents, which was segregated from the polymer prior to implantation. In some embodiments, the desired effect is manifested as a function of exposure to a component of the liposome contents, which was segregated from the polymer prior to implantation, for example, the component processes the agent of interest to its final form, for example, if a precursor is dispersed in the polymer solution, which is acted upon by an agent contained within the liposome, to form a desired therapeutic product. Such compounds are well known in the art and comprise desired proteins, such as cytokines, factors, such as coagulation factors, or others, as will be appreciated by one skilled in the art.

The scaffolding matrices of this invention comprise liposomes encapsulating cross-linking agents, which are segregated from the polymer, when the matrix is in an injectable form.

In one embodiment, the injectable form of the scaffolding material is kept at a first temperature, which is less than the phase transition temperature of the mixture of lipids, phospholipids, or combination thereof, of which the liposome is comprised. In one embodiment, the first temperature will range from about 4° C. to about 35° C. In one embodiment, upon injection, the scaffolding material is exposed to a higher temperature, which is the same or greater than the phase transition temperature of the mixture of lipids, thereby affecting the segregation of the cross-linking agent and polymer in the scaffolding material. Such a liposome is referred to herein as “thermo-responsive”, in some embodiments, owing to the temperature conditions at which the material is maintained, and its effect on segregation of the encapsulated cross-linker/active agent, and the polymers of the scaffold material.

In some embodiments, the term “thermo-responsive” refers to a single degree of temperature, or in some embodiments, a range of temperatures, whereupon if the lipsome is exposed to such temperature or range of temperature, the liposome architecture is affected, or in some embodiments, becomes permeable, or in some embodiments, enables de-segregation of previously segregated componenets, as herein described.

In one embodiment, the thermo-responsive liposome comprises a mixture of lipids, phospholipids, or a combination thereof. In one embodiment, the lipid is cholesterol. In another embodiment, the phospholipid is a dipalmitoyl-phosphatidylcholine (DPPC), dimyristoylphosphatidylcholine (DMPC), distearoyl-phosphatidylcholine (DSPC), dipalmitoyl-phosphatidylethanolamine (DPPE), dimyristoylphosphatidylethanolamine (DMPE), distearoyl-phosphatidylethanolamine (DSPE) or a combination thereof. In one embodiment, the phospholipid is glycerol-based, such as, for example, 1,2-dimyristoyl-sn-glycero-3-phosphocholine (DMPC), 1,2-dipentadecanoyl-sn-Glycero-3-Phosphocholine, 1,2-diheptadecanoyl-sn-Glycero-3-Phosphocholine, 1-myristoyl-2-palmitoyl-sn-Glycero-3-Phosphocholine, 1-myristoyl-2-stearoyl-sn-Glycero-3 -Phosphocholine, 1-palmitoyl-2-myristoyl-sn-Glycero-3-Phosphocholine, 1-stearoyl-2-myristoyl-sn-Glycero-3-Phosphocholine, 1-stearoyl-2-palmitoyl-sn-Glycero-3-Phosphocholine, 1,2-Dimyristoyl-sn-Glycero-3-[Phospho-rac-(1-glycerol)], 1,2-Dipalmitoyl-sn-Glycero-3-[Phospho-rac-(1-glycerol)], 1,2-Dimyristoyl-sn-Glycero-3-[Phospho-L-Serine], 1,2-Diauroyl-sn-Glycero-3-Phosphate (Monosodium Salt), 1,2-Diauroyl-sn-Glycero-3-Phosphoethanolamine, 1,2-Dielaidoyl-sn-Glycero-3-Phosphoethanolamine, 1-Palmitoyl-2-Oleoyl-sn-Glycero-3-Phosphoethanolamine, a combination thereof, or a salt thereof.

In another embodiment, the phospholipids may comprise Phosphaditylcholine (PC), Phosphatidylethanolamine (PE), PEG-PE, Diacetylene Phospholipids, Hydrogenated Phospholipids, Phosphatidylserine (PS), Phosphatidylinositol (PI & PIP's), Phosphatidic Acid (PA), Phosphatidylglycerol (PG), a combination thereof or a salt thereof.

In some embodiments, the lipid may comprise Cardiolipin (CA), Diacylglycerides (DG), PEG Lipids, PEG Ceramide, Fatty Acid Modified Derivatives, Sphingolipids, Dolichols, Ether lipids, Oxidized lipids, Lysophosphatides, Lipid A, a combination thereof, or a salt thereof.

In one embodiment, the liposome comprises two or more phospholipids. In another embodiment, the liposome comprises 2 or more lipids. In another embodiment, the liposome comprises any combination of lipid and phospholipid, as will be appreciated by one skilled in the art.

In another embodiment, the relative concentration of one of said two or more phospholipids is varied, so as to increase permeability of said liposome. For example, as shown in FIG. 3 herein, varying the DPPC/DMPC weight ratio influenced the release profile of the encapsulated enzyme. As the fraction of DPPC increased, the overall phase transition temperature increased, which in turn affected the release rate of the encapsulated agent.

In some embodiments, the DPPC/DMPC weight ratio varies from 2:1-10:1, for example, in one embodiment, having a ratio of 14:6 or, in another embodiment, having a ratio of 15:5.

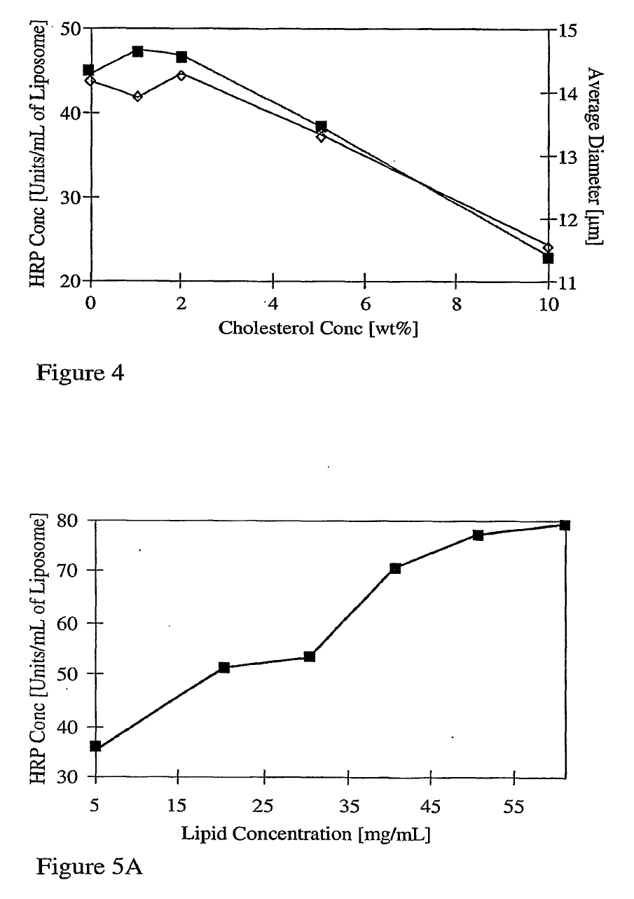

In another embodiment, the liposome further comprises a lipid, which inreases fluidity of the liposome. For example, as shown in FIG. 4 herein, adding cholesterol modified bilayer fluidity and may decrease encapsulated product leakage at room temperature.

Liposomes used in the scaffolding materials and methods of this invention encapsulate cross-linking agents. Cross-linking agents, for purposes of this invention, facilitate the formation of a covalent bond between 2 atoms. In one embodiment, the cross-linking agent may comprise a zero-length cross-linking agent.

In one embodiment, the cross-linking agent is (1 ethyl 3-(3dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or a combination thereof.

In one embodiment, the cross-linking agent is a homobifunctional cross-linker, such as, for example, N-hydroxysuccinimide esters (e.g. disuccinimidyl suberate or dithiobis(succinimidylpropionate), homobifunctional Irnidoesters (e.g. dimethyladipimidate or dimethyl pimelimidate), Sulfhydryl-reactive crosslinkers (e.g. 1,4di-[3′-(2′-pyridyldithio)propionamido]butane), Difluorobenzene derivatives (e.g. 1,5-difluoro-2,4-dinitrobenzene), Aldehydes (e.g. formaldehyde, glutaraldehyde), Bis-epoxides (e.g. 1,4-butanediol is diglycidyl ether), Hydrazides (e.g. adipic acid dihydrazide), Bis-diazonium derivatives (e.g. o-tolidine), Bis-alkylhalides, or a combination thererof.

In one embodiment, the cross-linking agent is a heterobifunctional cross-linker, such as, for example, an amine-reactive and sulfhydryl-reactive crosslinker (e.g. N-succinimidyl 3-(2-pyridyldithio)propionate, a carbonyl-reactive and Sulfhydryl-reactive crosslinker (e.g. 4-(4N-maleimidophenyl)butyric acid hydrazide), or a combination thereof.

In some embodiments, the cross-linking agent is a trifunctional cross-linkers, such as, for example, 4-Azido-2-nitrophenylbiocytin-4-nitrophenyl ester, sulfosuccinimidyl-2-[6-biotinamido]-2-(p-azidobenzamido)hexanoamido]ethyl-1,3′-dithiopropionate (sulfo-SBED), or a combination thereof.

In another embodiment, the cross-linking agent is an enzyme, which in one embodiment, is a transglutaminase, peroxidase, xanthine oxidase, polymerase, ligase, or a combination thereof.

In other embodiments, the cross-linking agent is any known in the art, including non-zero length cross-linking agents, which may exhibit thermo-responsiveness, in the materials, processes and methods of this invention.

The choice of concentration of the cross-linking agent utilized for activity will vary, as a function of the volume, agent and polymer chosen, in a given application, as will be appreciated by one skilled in the art.

In some embodiments, the encapsulated agent is a molecule which stimulates polymerization of a desired monomer to a polymer, or in another embodiment, elongation of a desired polymer. In one embodiment, an injectable scaffold material of this invention comprises liposome encapsulated thrombin, dispersed in a liquid comprising fibrinogen, and other factors necessary for clot formation. In some embodiments, such injectable scaffolds can be administered to subjects with wounds, who suffer from clotting disorders of any kind. For example, and in some embodiments, the subject may have a genetic mutation, which results in reduced or abnormal production of a requisite clotting factor. According to this aspect of the invention, such a factor may be encapsulated in a liposome, as part of an injectable scaffolding material as herein described, where all other factors are present in the liquid comprising the polymer, in this case fibrinogen. Upon administration to the subject, the scaffolding material fills the region of the wound, fibrinogen polymerizes, etc., and a clot is formed at the desired site.

It is to be understood that this invention provides for any material(s), which comprises a thermo-sensitive liposome encapsulated cross-linker, dispersed in a polymer solution, which allows for the segregation of the encapsulated cross-linker and the polymer solution, and wherein additional agents of interest may be incorporated in the liposome or polymer solution, as will be appreciated by one skilled in the art.

In some embodiments, the terms “a”, “an”, and “the” include plural references, and can be taken to refer to at least one, or two or more, or as many as applicable for the referenced material or indication. Similarly, reference to the singular includes a reference to the plural, with regard to any element as described herein.

In another embodiment, the materials are formed without the use of organic solvents, and as a result many compounds, which may be denatured in the presence of organic solvents, may be incorporated in the scaffolding materials of this invention, without any untoward effects due to the solvent. For example, protein denaturation by organic solvents is a well-recognized phenomenon. Protein incorporation within the scaffolding materials of this invention would, in one embodiment, not be subject to such denaturation. Such a phenomenon may also be important for the incorporation of molecules involved in tissue engineering, repair or remodeling, for example, enzymes, such as matrix metallo proteinases growth factors, such as bone morphogenetic proteins, cytokines, chemokines, and related molecules, or controlled delivery of such agents, as described herein.

In another embodiment, the materials of this invention may comprise other additives, such as, for example, pigments, dyes, organic or inorganic fibrous or particulate reinforcing or extending fillers, thiotropic agents, indicators, inhibitors or stabilizers (weathering or non-yellowing agents), surfactants, flow aids, chain transfer agents, foaming agents, porosity modifiers, and the like, sugars, cyclic sugars, cyclodextrins, synthetic derivatives of cyclodextrins, glycolipids, glycosaminoglycans, lipids, amino acids (e.g.; but not limited to: glycine, sodium glutamate, proline, α-alanine, β-alanine, lysine-HCl, 4-hydroxyproline), peptides and polypeptides, proteins, amines (e.g.; but not limited to: betaine, trimethylamine N-oxide), lipo-proteic molecules, polyols, gums, waxes, antioxidants, anti-reductants, buffering agents, inorganic and organic salts (e.g; but not limited to: ammonium, sodium, and magnesium sulfate, potassium phosphate, sodium fluoride, sodium acetate, sodium polyethylene, sodium caprylate, propionate, lactate, succinate), radical scavengers, diluents (e.g.; but not limited to: mannitol, lactose, sorbitol, sucrose, inositol, dicalcium phosphate, calcium sulfate, cellulose, hydroxypropylmethylcellulose, kaolin, sodium chloride, starch), cryoprotectants, and natural or synthetic polymers, a binder (e.g.;

but not limited to: starch; gelatin; sugars as sucrose, glucose, dextrose, molasses, and lactose;

natural and synthetic gums such as acacia, sodium alginate, extract of Irish moss, panwar gum, ghatti gum, mucilage of isapol husks, carboxymethylcellulose, methylcellulose, hydroxypropyl methylcellulose, hydroxylpropyl cellulose, ethyl cellulose, polyvinylpyrrolidone, Veegum, larch arabogalactan; polyethylene glycols; ethylcellulose; waxes; water and achools, amylase, methacrylate and methyl methacrylate copolymers), plasticizer (e.g; but not limited to: glycerin, propylene glycol, polyethylene glycols, triacetin, acetylated monoglyceride, citrate esters, phthalate esters), disaggregant (e.g.; but not limited to: starches, clays, celluloses, algins, gums, cross-linked natural and synthetic polymers, Veegum HV, methylcellulose, agar, bentonite, cellulose and wood products, natural sponge, cation-exchange resins, alginic acid, guar gum, citrus pulp, carboxymethylcellulose, combinations of sodium lauryl sulfate and starch) used in any of their physical or processed state. Any derivative of above-mentioned molecules are included as well.

The scaffolding material of this invention may further comprise a compound of interest. Such scaffolding material may be utilized, in some embodiments, as a means delivery of the compound of interest to a desired site in a subject.

In one embodiment, the term “compound of interest”, as used anywhere herein, refers to any desired molecule, and may comprise, inter alia, a nucleic acid, a hormone, a growth factor, a cytokine, a chemokine, a bone morphogenetic protein, a matrix metallo-proteinases, a peptide, a drug, an enzyme or a combination thereof.

The term “compound” and “drug” and “agent” are to be considered synonymous, when referred to herein, and represent a molecule whose incorporation within the materials of this invention, or whose delivery to a site in a subject, is desired. In one embodiment, the agent is incorporated directly within the materials of this invention or, in another embodiment, the agent is incorporated within the materials of this invention, following its modification, as a means of enhancing the stability of the compound, or processing of the compound, or dilution of the compound, including in situ dilution of the compound, or combinations thereof.

In another embodiment, the compound is part of a formulation which is incorporated within the materials of this invention In one embodiment, compounds for use in the materials and/or methods of this invention may comprise, inter-alia, an antibody or antibody fragment, a peptide, an oligonucleotide, a ligand for a biological target, an immunoconjugate, a chemomimetic functional group, a glycolipid, a labelling agent, an enzyme, a metal ion chelate, an enzyme cofactor, a cytotoxic compound, a bactericidal compound, a bacteriostatic compound, a chemotherapeutic, a growth factor, a hormone, a cytokine, a toxin, a prodrug, an antimetabolite, a microtubule inhibitor, a radioactive material, a targeting moiety, or any combination thereof.

In one embodiment, the term “antibody or antibody fragment” refers to intact antibody molecules as well as functional fragments thereof, such as Fab, F(ab′)2, and Fv that are capable of binding to an epitope. In one embodiment, an Fab fragment refers to the fragment which contains a monovalent antigen-binding fragment of an antibody molecule, which can be produced by digestion of whole antibody with the enzyme papain to yield an intact light chain and a portion of one heavy chain. In one embodiment, Fab′ fragment refers to a part of an antibody molecule that can be obtained by treating whole antibody with pepsin, followed by reduction, to yield an intact light chain and a portion of the heavy chain. Two Fab′ fragments may be obtained per antibody molecule. In one embodiment, (Fab′)2 refers to a fragment of an antibody that can be obtained by treating whole antibody with the enzyme pepsin without subsequent reduction. In another embodiment, F(ab′)2 is a dimer of two Fab′ fragments held together by two disulfide bonds. In one embodiment, Fv, may refer to a genetically engineered fragment containing the variable region of the light chain and the variable region of the heavy chain expressed as two chains. In one embodiment, the antibody fragment may be a single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

In one embodiment, the materials may comprise a peptide. In some embodiments, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomnimetic compounds are well known in the art and are specified, for example, in Quantitative Drug Design, C.A. Ramsden Gd., Chapter 17.2, F. Choplin Pergamon Press (1992), which is incorporated by reference as if fully set forth herein.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

In one embodiment, the material comprises an oligonucleotide, a nucleic acid, or a vector. In some embodiments, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

The materials of this invention may comprise nucleic acids, in one embodiment, or in another embodiment, the methods of this invention may include delivery of the same, wherein, in another embodiment, the nucleic acid is a part of a particular vector. In one embodiment, polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding the protein or peptide of interest, or fragments thereof, retaining wild type function exhibiting the same biological effect, despite these variations. Each of these represents a separate embodiment of this present invention.

The nucleic acids can be produced by any synthetic or recombinant process such as is well known in the art. Nucleic acids can further be modified to alter biophysical or biological properties by means of techniques known in the art. For example, the nucleic acid can be modified to increase its stability against nucleases (e.g., “end-capping”), or to modify its solubility, or binding affinity to complementary sequences.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention.

In one embodiment, the agent is one which may inhibit gene expression in a subject. In one embodiment, the agent that inhibits gene expression, activity or function comprises a nucleic acid. The nucleic acid may, in one embodiment, be DNA, or in another embodiment, the nucleic acid is RNA. In other embodiments, the nucleic acid may be single or double stranded.

In one embodiment, the agents used in the materials and/or methods of this invention may be used for gene silencing applications. In one embodiment, the activity or function of a particular gene is suppressed or diminished, via the use of antisense oligonucleotides. In one embodiment, the antisense molecules may be conjugated to the polymers of this invention. Inhibition of gene expression, activity or function is effected, in another embodiment, via the use of small interfering RNAs, which provides sequence-specific inhibition of gene expression for example, as described in Elbashir S M, et al (2001) Nature 411:494-498; Fire et al. (1998) Nature 391: 806-11; Waterhouse, P. M., et al. (1998). Proc. Natl. Acad. Sci. USA 95, 13959-13964 and Wang, Z., et al. (2000). J. Biol. Chem. 275, 40174-40179.

The above nucleic acids may be delivered to any tissue or cells in one embodiment, in their native form, or, in another embodiment within an expression vector that is competent to transfect cells in vitro and/or in vivo, and comprise an embodiment of this invention. In some embodiments, such transfected, transduced or transformed cells, may be incorporated into scaffolding material of this invention, so that engineered cells may comprise the scaffolding of this invention.

In one embodiment, the nucleic acid encodes for an antibacterial, antiviral, antifungal or antiparasitic peptide or protein. In another embodiment, the nucleic acid encodes for a peptide or protein with cytotoxic or anti-cancer activity. In another embodiment, the nucleic acid encodes for an enzyme, a receptor, a channel protein, a hormone, a cytokine, a bone morphogenetic protein, a matrix metallo-proteinase, or a growth factor. In another embodiment, the nucleic acid encodes to for a peptide or protein, which is immunostimulatory. In another embodiment, the nucleic acid encodes for a peptide or protein, which inhibits inflammatory or immune responses.

It is to be understood that the methods of this invention also provide for the incorporation of cells within the materials of this invention, whereby cells may be affected by molecules within the materials, or in another embodiment, the cells may take up the incorporated molecule, or a combination thereof. For example, a chemokine or inflammatory stimulus may be incorporated within a matrix of this invention, which serves to recruit a cell of interest, which may then take up, for example, a vector which is also incorporated within the matrix, and whereby the cell, following expression of a molecule encoded within the vector may perform a desired effector function. Such methods may be considered as part of cell therapy, as well as other variations of the same concept, whereby multiple components incorporated within a matrix of this invention may specifically stimulate a particular population or populations of cells, and may promote a specific function of these cells thereby.

In one embodiment, specific cells are recruited to the materials, or matrices of this invention or incorporated within the materials or matrices of this invention. In one embodiment, the cell may be any desired cell, such as, in one embodiment, an epithelial cell, an airway epithelial cell (e.g.; bronchial epithelial cell, small airway epithelial cell), an endothelial cell (e.g.; cell obtained from dermal microvasculature, lung microvasculature, uterine rnicrovasculature, umbilical vein, coronary artery, pulmonary artery, aorta, iliac artery umbilical artery), a dermal or lung fibroblast, an epidermal keratinocyte, an epidermal melanocite cell, a smooth muscle cell (e.g.; cell obtained from aorthic smooth muscle, bronchial smooth muscle, coronary artery, pulmonary artery, umbelical artery, uterine smooth muscle), skeletal muscle cell, dendritic cells, peripheral blood mononuclear cell, a lung cell, a renal cell (e.g.; cell obtained from proximal tubule, cortical epithelia, renal epithelia, or a mesangial cell), a liver cell, a cardiocyte, a neural cell, an astrocyte, a neural progenitor cell, a glial cell, a prostate ephitelial cell, a prostate stromal cell, a professional antigen presenting cell, a lymphocyte, an M cell, a macrophage, a granulocyte, erythroid progenitor cell, a pancreatic cell, a stem cell, mesenchymal stem cell, hematopoictic stem cell, cell derived (with any procedure) from bone marrow, cell derived (with any procedure) from umbilical cord blood, a myoblast, a megakaryocyte, a hepatocyte, an osteoblast, an osteocyte, an osteoclast, a chondrocyte, a chodroblast, or other bone or cartilage cells and may be used for applications as described in, for example, Wilson, J. M et al. (1988) Proc. Natl. Acad. Sci. USA 85:3014-3018; Armentano, D. et al. (1990) Proc. Natl. Acad. Sci. USA 87:6141-6145; Wolff, J. A. et al. (1990) Science 247:1465-1468; Chowdhury, J. R. et al. (1991) Science 254:1802-1805; Ferry, N. et al. (1991) Proc. Natl. Acad. Sci. USA 88:8377-8381; Wilson, J. M. et al. (1992) J. Biol. Chem. 267:963-967; Quantin, B. et al. (1992) Proc. Natl . Acad. Sci. USA 89:2581-2584; Dai, Y. et al. (1992) Proc. Natl. Acad. Sci. USA 89:10892-10895; van Beusechem, V. W. et al. (1992) Proc. Natl. Acad Sci. USA 89:7640-7644; Rosenfeld, M. A. et al. (1992) Cell 68:143-155; Kay, M. A. et al. (1992) Human Gene Therapy 3:641-647; Cristiano, R. J. et al. (1993) Proc. Natl. Acad Sci. USA 90:2122-2126; Hwu, P. et al. (1993) J. Immunol. 150:4104-4115; and Herz, J. and Gerard, R. D. (1993) Proc. Natl. Acad Sci. USA 90:2812-2816. In some emboidents, recruitment is of platelets is similarly accomplished.

Recruitment of desired cells may be accomplished, in one embodiment, via the incorporation of a specific ligand for a particular receptor expressed on the desired cell surface, or a particular composition of the matrix, for example, enriched for specific components of extracellular matrix, etc., as will be appreciated by one skilled in the art.

In one embodiment, the materials/matrices of this invention may further comprise a “drug” or “compound” or “agent”, which refers in some embodiments, to a substance applicable for use in the diagnosis, or in another embodiment, cure, or in another embodiment, mitigation, or in another embodiment, treatment, or in another embodiment, prevention of a disease, disorder, condition or infection. In one embodiment, the “drug” or “compound” or “agent” for use in the scaffolding materials and/or methods this invention, refers to any substance which affects the structure or function of the target to which it is applied.

In another embodiment, the “drug” or “compound” or “agent” for use in the materials/matrices and/or methods of this invention, is a molecule that alleviates a symptom of a disease or disorder when administered to a subject afflicted thereof. In one embodiment, the “drug” or “compound” or “agent” for use in materials/matrices and/or methods of this invention is a synthetic molecule, or in another embodiment, a naturally occurring compound isolated from a source found in nature.

In one embodiment, the “drug” or “compound” or “agent” for use in the materials/matrices and/or methods of this invention may comprise antihypertensives, antidepressants, antianxiety agents, anticlotting agents, anticonvulsants, blood glucose-lowering agents, decongestants, antihistamines, histamine, antitussives, anti-inflammatories, antipsychotic agents, cognitive enhancers, cholesterol-reducing agents, antiobesity agents, autoimmune disorder agents, anti-impotence agents, antibacterial and antifungal agents, hypnotic agents, anti-Parkinsonism in agents, antibiotics, antiviral agents, anti-neoplastics, barbituates, sedatives, nutritional agents, beta blockers, emetics, anti-emetics, diuretics, anticoagulants, cardiotonics, androgens, corticoids, anabolic agents, growth hormone secretagogues, anti-infective agents, coronary vasodilators, carbonic anhydrase inhibitors, antiprotozoals, gastrointestinal agents, serotonin antagonists, anesthetics, hypoglycemic agents, dopaminergic agents, anti-Alzheimer's Disease agents, anti-ulcer agents, platelet inhibitors and glycogen phosphorylase inhibitors, insulin, diagnostic markers, drugs used for the control of birth, natural products, calcyfying agents, cell mediators, cell inhibitors, antimitotic agents, alkylating agents, immunomodulators, analgesics, vaccines, sympathomimetic agents, cholinomimetic agents, adrenergic and adrenergic neuron blocking agent, antimuscarinic and antispasmodic agents, skeletal muscle relaxant, anti-migrane agents, central nervous system stimulans, immunosuppressive agents, vitamins, parasiticides, drugs for the treatment of ipo-/iper-tyroidism, osteoporosis, osteopetrosis, arthritis, epilepsy, glaucoma and eye diseases.

In one embodiment, examples of the “drug” or “compound” or “agent” for use in the materials/matrices and/or methods of this invention comprise, inter-alia, antihypertensives including prazosin, nifedipine, trimazosin, amlodipine, and doxazosin mesylate; the antianxiety agent hydroxyzine; a blood glucose lowering agent such as glipizide; an anti-impotence agent such as sildenafil citrate; anti-neoplastics such as chlorambucil, lomustine or echinomycin; anti-inflammatory agents such as betamethasone, prednisolone, piroxicam, aspirin, flurbiprofen and (+)-N-{4-[3-(4-fluorophenoxy)phenoxy]-2-cyclopenten-1-yl}-N-hyroxyurea; antivirals such as acyclovir, nelfinavir, or virazole; vitamins/nutritional agents such as retinol and vitamin E; emetics such as apomorphine; diuretics such as chlorthalidone and spironolactone; an anticoagulant such as dicumarol; cardiotonics such as digoxin and digitoxin; androgens such as 17-methyltestosterone and testosterone; a mineral corticoid such as desoxycorticosterone; a steroidal hypnotic/anesthetic such as alfaxalone; an anabolic agent such as fluoxymesterone or methanstenolone; antidepression agents such as fluoxetine, pyroxidine, venlafaxine, sertraline, paroxetine, sulpiride,[3,6-dimethyl-2-(2,4,6-trimethyl-phenoxy)-pyridin-4-yl]-(lethylpropyl)-amine or 3,5-dimethyl-4-(3′-pentoxy)-2-(2′,4′,6′-trimethylphenoxy)pyridine; an antibiotic such as ampicillin and penicillin G or belonging to the family of penicillines, cephalosporins, aminoglycosidics, macrolides, carbapenem and penem, beta-lactam monocyclic, inhibitors of beta-lactamases, tetracyclins, polipeptidic antibiotics, chloramphenicol and derivatives, fusidic acid, lincomicyn, novobiocine, spectinomycin, poly-etheric ionophores, quinolones; an anti-infective such as benzalkonium chloride or chlorhexidine; a coronary vasodilator such as nitroglycerin or mioflazine; a hypnotic such as etornidate; a carbonic anhydrase inhibitor such as acetazolamide or chlorzolamide; an antifungal such as econazole, terconazole, fluconazole, voriconazole or griseofulvin; an antiprotozoal such as metronidazole; an imidazole-type anti-neoplastic such as tubulazole; an anthelmintic agent such as thiabendazole or oxfendazole; an antihistamine such as astemizole, levocabastine, cetirizine, or cinnarizine; a decongestant such as pseudoephedrine; antipsychotics such as fluspirilene, penfluridole, risperidone or ziprasidone; a gastrointestinal agent such as loperamide or cisapride; a serotonin antagonist such as ketanserin or mianserin; an anesthetic such as lidocaine; a hypoglycemic agent such as acetohexamide; an anti-emetic such as dimenhydrinate; an antibacterial such as cotrimoxazole; a dopaminergic agent such as L-DOPA; anti-Alzheimer agents such as THA or donepezil; an anti-ulcer agent/H2 antagonist such as famotidine; a sedative/hypnotic such as chlordiazepoxide or triazolam; a vasodilator such as alprostadil; a platelet inhibitor such as prostacyclin; an ACE inhibitor/antihypertensive such as enalaprilic acid or lisinopril; a tetracycline antibiotic such as oxytetracycline or minocycline; a macrolide antibiotic such as azithromycin, clarithromycin, erythromycin or spiramycin; and glycogen phosphorylase inhibitors such as [R-(R*S-*)]-5-chloro-N-[2-bydroxy-3[methoxymethylamino]-3-oxo-1-(phenylmethyl)-propyl]-IH-indole-2-carboxamide or 5-chloro-1-Hindole-2-carboxylic acid [(IS)-benzyl(2R)-hydroxy-3-((3R,4S)dihydroxy-pyrrolidin-1-yl-)-oxypropyl] amide.

Further examples of the “drug” or “compound” or “agent” for use in the materials/matrices and/or methods of this invention are the glucose-lowering drug chlorpropamide, the anti-fungal fluconazole, the anti-hypercholesterolemic atorvastatin calcium, the antipsychotic thiothixene hydrochloride, the anxiolytics hydroxyzine hydrochloride or doxepin hydrochloride, the anti-hypertensive amlodipine besylate, the antiinflammatories piroxicam and celicoxib and valdicoxib, and the antibiotics carbenicillin indanyl sodium, bacampicillin hydrochloride, troleandomycin, and doxycycline hyclate.

In another embodiment a “drug” or “compound” or “agent” for use in the materials/matrices and/or methods of this invention may comprise other antineoplastic agents such as platinum compounds (e.g., spiroplatin, cisplatin, and carboplatin), methotrexate, fluorouracil, adriamycin, mitomycin, ansamitocin, bleomycin, cytosine arabinoside, arabinosyl adenine, mercaptopolylysinie, vincristine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, paclitaxel and other taxenes, rapamycin, manumycin A, TNP-470, plicamycin (mithramycin), aminoglutethimide,estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, interferon .alpha.-2a, interferon .alpha.-2b, teniposide (VM-26), vinblastine sulfate (VLB), vincristine sulfate, bleomycin sulfate, hydroxyurea ,procarbazine, and dacarbazine; mitotic inhibitors such as etoposide, colchicine, and the vinca alkaloids, radiopharmaceuticals such as radioactive iodine and phosphorus products; hormones such as progestins, estrogens and antiestrogens; anti-helmintics, antimalarials, and antituberculosis drugs; biologicals such as immune serums, antitoxins and antivenoms; rabies prophylaxis products; bacterial vaccines; viral vaccines; respiratory products such as xanthine derivatives theophylline and arninophylline; thyroid agents such as iodine products and anti-thyroid agents; cardiovascular products including chelating agents and mercurial diuretics and cardiac glycosides; glucagon; blood products such as parenteral iron, hemin, hematoporphyrins and their derivatives; biological response modifiers such as muramyldipeptide, muramyltripeptide, microbial cell wall components, lymphokines (e.g., bacterial endotoxin such as lipopolysaccharide, macrophage activation factor), sub-units of bacteria (such as Mycobacteria, Corynebacteria), the synthetic dipeptide N-acetyl-muramyl-L-alanyl-D-isoglutamine; anti-fungal agents such as ketoconazole, nystatin, griseofulvin, flucytosine (5-fc), miconazole, amphotericin B, ricin, cyclosporins, and β-lactam antibiotics (e.g., sulfazecin); hormones such as growth hormone, melanocyte stimulating hormone, estradiol, beclomethasone dipropionate, betamethasone, betamethasone acetate and betamethasone sodium phosphate, vetamethasoine disodium phosphate, vetamethasone sodium phosphate, cortisone acetate, dexamethasone, dexamethasone acetate, dexamethasone sodium phosphate, flunisolide, hydrocortisone, hydrocortisone acetate, hydrocortisone cypionate, hydrocortisone sodium phosphate, hydrocortisone sodium. succinate, methylprednisolone, methylprednisolone acetate, methylprednisolone sodium succinate, paramethasone acetate, prednisolone, prednisolone acetate, prednisolone sodium phosphate, prednisolone tebutate, prednisone, triamcinolone, triamcinolone acetonide, triamcinolone diacetate, triamcinolone hexacetonide, fludrocortisone acetate, oxytocin, vassopressin, and their derivatives; vitamins such as cyanocobalamin neinoic acid, retinoids and derivatives such as retinol palmitate, and .alpha.-tocopherol; peptides, such as manganese super oxide dismutase; enzymes such as alkaline phosphatase; anti-allergic agents such as amelexanox; anti-coagulation agents such as phenprocoumon and heparin; circulatory drugs such as propranolol; metabolic potentiators such as glutathione; antituberculars such as para-aminosalicylic acid, isoniazid, capreomycin sulfate cycloserine, ethambutol hydrochloride ethionamide, pyrazinamide, rifampin, and streptomycin sulfate; antivirals such as amantadine azidothymidine (AZT, DDI, Foscarnet, or Zidovudine), ribavirin and vidarabine monohydrate (adenine arabinoside, ara-A); antianginals such as diltiazem, nifedipine, verapamil, erythritol tetranitrate, isosorbide dinitrate, nitroglycerin (glyceryl trinitrate) and pentaerythritol tetranitrate;

anticoagulants such as phenprocoumon, heparin; antibiotics such as dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin including penicillin G and penicillin V, ticarcillin rifampin and tetracycline; antiinflammatories such as diflunisal, ibuprofen, indomethacin, meclofenamate, mefenamic acid, naproxen, oxyphenbutazone, phenylbutazone, piroxicam, sulindac, tolmetin, aspirin and salicylates; antiprotozoans such as chloroquine,hydroxychloroquine, metronidazole, quinine and meglumine antimonate; antirheumatics such as penicillamine; narcotics such as paregoric;opiates such as codeine, heroin, methadone, morphine and opium; cardiac glycosides such as deslanoside, digitoxin, digoxin, digitalin and digitalis; neuromuscular blockers such as atracurium mesylate, gallamine triethiodide, hexafluorenium bromide, metocurine iodide, pancuronium bromide, succinylcholine chloride (suxamethonium chloride), tubocurarine chloride and vecuronium bromide; sedatives (hypnotics) such as amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride, paraldehyde, pentobarbital, pentobarbital sodium, phenobarbital sodium, secobarbital sodium, talbutal, temazepam and triazolam; local anesthetics such as bupivacaine hydrochloride, chloroprocaine hydrochloride, etidocaine hydrochloride, lidocaine hydrochloride, mepivacaine hydrochloride, procaine hydrochloride and tetracaine hydrochloride; general anesthetics such as droperidol, etomidate, fentanyl citrate with droperidol, ketamine hydrochloride, methohexital sodium and thiopental sodium; and radioactive particles or ions such as strontium, iodide rhenium and yttrium.

In one embodiment, the “drug” or “compound” or “agent” for use in the materials/matrices and/or methods of this invention is a therapeutic compound. In one embodiment, the therapeutic compound is a peptide, a protein or a nucleic acid. In another embodiment, the therapeutic compound is an antibacterial, antiviral, antifungal or antiparasitic compound. In another embodiment, the therapeutic compound has cytotoxic or anti-cancer activity. In another embodiment, the therapeutic compound is an enzyme, a receptor, a channel protein, a hormone, a cytokine or a growth factor. In another embodiment, the therapeutic compound is immunostimulatory. In another embodiment, the therapeutic compound inhibits inflammatory or immune responses.

In one embodiment, the term “therapeutic”, refers to a molecule, which when provided to a subject in need, provides a beneficial effect. In some cases, the molecule is therapeutic in that it functions to replace an absence or diminished presence of such a molecule in a subject. In one embodiment, the molecule is a nucleic acid coding for the expression of a protein is absent, such as in cases of an endogenous null mutant being compensated for by expression of the foreign protein. In other embodiments, the endogenous protein is mutated, and produces a non-functional protein, compensated for by the expression of a heterologous functional protein. In other embodiments, expression of a heterologous protein is additive to low endogenous levels, resulting in cumulative enhanced expression of a given protein. In other embodiments, the molecule stimulates a signalling cascade that provides for expression, or secretion, or others of a critical element for cellular or host functioning.

In another embodiment, the therapeutic molecule may be natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deamninases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the a family, transforming growth factors of the , family, gastrins, secretins, cholecystokinins, somatostatins, serotonins, substance P, transcription factors or combinations thereof.

In another embodiment, this invention also comprises incorporation of any toxic substance for therapeutic purpose. In one embodiment, the materials/matrices of this invention may incorporate an oligonucleotide encoding a suicide gene, which when in contact with diseased cells or tissue, is expressed within such cells. In one embodiment, the term “suicide gene” refers to a nucleic acid coding for a product, wherein the product causes cell death by itself or in the presence of other compounds. A representative example of a suicide gene is one, which codes for thymidine kinase of herpes simplex virus. Additional examples are thymidine kinase of varicella zoster virus and the bacterial gene cytosine deaminase, which can convert 5-fluorocytosine to the highly cytotoxic compound 5-fluorouracil.

Suicide genes may produce cytotoxicity by converting a prodrug to a product that is cytotoxic. In one embodiment, the term “prodrug” means any compound that can be converted to a toxic product for cells. Representative examples of such a prodrug is gancyclovir which is converted in vivo to a toxic compound by HSV-thymidine kinase. The gancyclovir derivative subsequently is toxic to cells. Other representative examples of prodrugs include acyclovir, FIAU [1-(2-deoxy-2fluoro-β-D-arabinofuranosyl)-5-iodouracil], 6-methoxypurine arabinoside for VZV-TK, and 5-fluorocytosine for cytosine deaminase.

In another embodiment, the cytotoxic agent may comprise any agent that is detrimental to cells, such as, for example, taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof.

In another embodiment, the agent may be a radioactive agent, which in other embodiments, may include any radioisotope which is known in the art, used for example in diagnosing cancer, or in anti-tumor applications. Examples include, but are not limited to, indium-111, cobalt-60. Additionally, naturally occurring radioactive elements such as uranium, radium, and thorium which typically represent mixtures of radioisotopes, are suitable examples of a radioactive agent. In another embodiment, magnetic particles may be thus used, such as, for example, magnetic iron oxide particles. The metal ions are typically chelated with an organic chelating moiety.

In some embodiments, materials/matrices are provided at a site of wound in a subject, whereby the materials/matrices comprise cytotoxic substances, which serve to destroy or diminish the amount of diseased cells or tissue at the wound site, and in some embodiments, concurrently comprise tissue-promoting materials to selectively promote formation of healthy tissue. For example, and in one embodiment, various antimicrobial compounds can be incoporated in infected bone tissue, which serves to kill the source of infection. The same scaffolding material, according to this embodiment, may comprise stem cells and bone morphogenetic proteins, which in turn promote new bone formation, which may replace bone tissue damaged as a result of infection.

In one embodiment, the compounds, agents, cells of which the materials/matrices of this invention may be comprised, are interspersed within the materials/matrices. In one embodiment, the term interspersed refers to a compound which is free, or in another embodiment, conjugated, or in another embodiment, adsorbed on, or in another embodiment, coated, or in another embodiment, entrapped, or in another embodiment, encasulated within the scaffolding material of this invention.

In one embodiment, the materials/matrices further comprise cells, which, in another embodiment, are stem or progenitor cells.

In some embodiments, the invention provides a cosmetic filler, drug delivery vehicle or scaffold for tissue engineering or repair, comprising materials/matrices of this inveniton, as further described hereinbelow.

In another embodiment, this invention provides a process for preparing an injectable scaffolding material, comprising the steps of:
a. forming liposomes comprising a cross-linking agent encapsulated within a mixture of lipids, phopsholipids, or a combination thereof;
b. freezeing said lipsomes;
c. rehydrating said liposomes; and
d. dispersing said liposomes in a liquid comprising a polymer
   wherein said dispersing is conducted at a temperature which is less than the phase transition temperature of said mixture and said cross-linking agent or reactive species and said polymer are segregated at said temperature.

In one embodiment, the freezing is accomplished at a rate of between 0.5-350° C. per minute. In one embodiment, the freezing is accomplished at a rate of between 0.5-100° C. per minute. In one embodiment, the freezing is accomplished at a rate of between 10-250° C. per minute. In one embodiment, the freezing is accomplished at a rate of between 50-350° C. per minute.

In one embodiment, freezing is by any means, and using any device which may be used for such purpose, including, but not limited to temperature-regulated freezers, flash or immersion freezing in dry ice baths, liquid nitrogen, etc.

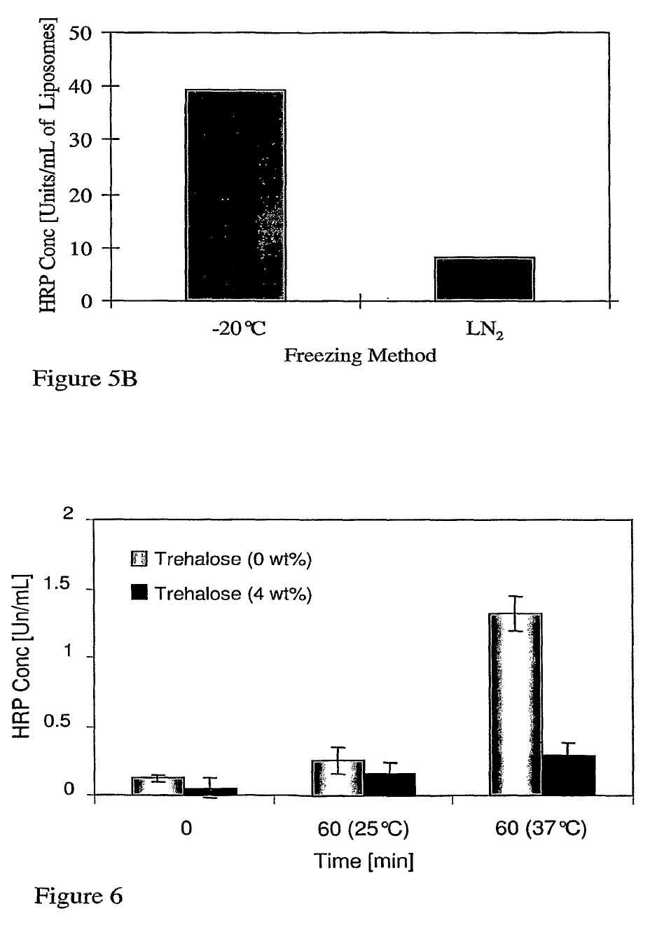

In some embodiments, freezing conditions can be tailored to optimize the activity and/or release rate of the material encapsulated. For example, FIG. 5 herein demonstrates that the rate of cooling affected the amount of HRP released from the liposomes. In some embodiments, a cryoprotectant may be added during lipsomal encapsulation of the cross-linker or active agent.

In one embodiment, the method further comprises the step of sonicating said mixture prior to freezing, in step (b). Sonicating the liposomes prior to freezing, as exemplified herein, may increase the encapsulation of the cross-linker (FIG. 8). In one embodiment, a mechanism whereby sonication achieves this result is via its ability to break up liposomes, convert multi-lamellar vesicles (MLV's) to single unilamellar vesicles (SUV's) and thereby affect the manner in which the liposomes re-form during rehydration step, thus influencing the total encapsulation.

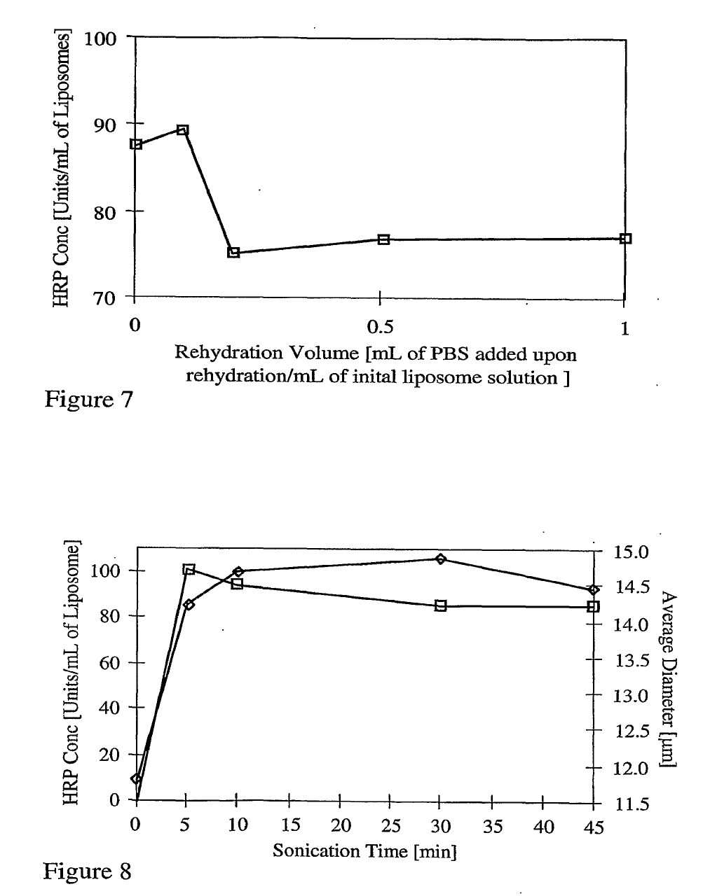

In some embodiments, the volume of solution used for rehydration is varied, which in turn may ultimately affect the amount of material released from the liposome, in the scaffolds of this invention (FIG. 7). In some embodiments, rehydrating is conducted with a buffered solution, which in some embodiments, is a salt solution comprising a monovalent or divalent cation. In one embodiment, the salt solution comprises sodium chloride, or salts of monovalent cations, or in another embodiment, calcium chloride, or salts of other divalent or trivalent cations In some embodiments, the rehydrating is conducted in a small or minimal volume, for example, less than 1 milliliter, or in some embodiments, less than half a milliliter, or in some embodiments, less than 400 μl, or in some embodiments, less that 300 μl, or in some embodiments, less that 275 μl, or in some embodiments, less that 250 μl, or in some embodiments, less that 200 μl, or in some embodiments, less that 175 μl, or in some embodiments, less that 150 μl, or in some embodiments, less that 125 μl, or in some embodiments, less that 100 μl, or in some embodiments, less that 75 μl, or in some embodiments, less that 50 μl.

As exemplified herein, rinsing liposomes following rehydration may affect liposomal stabilization, and thereby release of the cross-liner. In one embodiment, choice of buffer solution, and number, timing, etc. of the washes may be varied, in order to promote greater liposomal stabilization, and subsequent cross-linker, active agent release.

In some embodiments, other liposomal encapsulation methods may be utilized, to prepare the scaffolding materials of this invention. In some embodiments, such methods may comprise hydration in presence of enzyme at T>Tm to form multi-lamellar vesicles (MLVs), freeze-thaw cycles of MLVs (MLV-FAT), freeze-thaw cycles of MLVs followed by extrusion (FAT-VET), extrusion of MLVs followed by freeze-thaw cycles (VET-FAT), vesicles prepared by extrusion of MLVs (VET), sonication of MLVs to form single unilamellar vesicles (SUVs), homogenization with MicrofluidizerTm of MLVs to form MLV-MFVs, hydration and shearing lamellar phase to form multilamellar spherulites (MLS), interdigitation fusion method (IFV), extrusion of dehydration-rehydration vesicles (DRVs) (DRV-VET), microfluidization of DRVs to form (DRV-MFV), electroformed giant vesicles, reverse-phase evaporation method (REV), extrusion of REVs (REV-VET), ethanol injection method (VEI), vesicles prepared by water in oil in water emulsion process, detergent dialysis method (DDV), pro-liposome method (VPL), detergent-induced loading of lipid vesicles (DILL), super-critical liposome method, ether injection method, rapid solvent exchange method, bubble method, or others, as will be appreciated by one skilled in the art.

The methods of this invention have been exemplified herein, with materials/matrices comprising hyaluronic acid. By combining a hyaluronic acid-tyramine conjugated polymer (HA-Tyr) with thermoresponsive liposomes containing horseradish peroxidase (HRP), it was shown that solid material (e.g. scaffold) formation was controlled by temperature. The polymer/liposome solution remained as a liquid for several hours at room temperature, but was cured at body temperature within minutes. In vivo studies demonstrated herein, no interfering reactions inhibiting the release of the enzyme or the reaction between the polymer and cross-linker. Use of the scaffolding material was shown herein to serve as a substrate for tissue regeneration, representing an embodiment of this invention.

In another embodiment, this invention provides a method or tissue repair, regeneration, or organogenesis in a subject, the method comprising administering a material to a subject comprising;
∘ a polymer solution;
∘ a thermo-responsive liposome dispersed in said solution; and
∘ a zero-length cross-linking agent encapsulated in said liposome;
   wherein said polymer and said cross-linking agent are segregated at a first temperature, non-segregated at a second temperature, and said material is injectable at said first temperature.

According to this aspect of the invention, and in one embodiment, the material further comprises cells involved in tissue repair, regeneration, or organogenesis, as described herein, and may serve as a scaffolding material upon injection to the subject. In another embodiment, the material further comprises a tissue promoting factor, which in one embodiment, is a hormone, a nucleic acid, a growth factor, a chemokine, a peptide, an enzyme or a combination thereof.

According to this aspect of the invention, and in one embodiment, the material may be used for the production of tissue engineering cell supports (e.g.; scaffolds, barriers, membranes, tissue fillers). Such supports, in other embodiments, may be produced, or in another embodiment, used, with cells, or in another embodiment without cells, in combination with drugs, or in another embodiment, without additional compounds.

It is to be understood that numerous applications of the material of this invention are envisioned, such as, for example, in wound healing, tissue engineering, cell therapy, gene therapy, and others, as will be appreciated by one skilled in the art, and any application of a material of this invention, in any shape or form, is to be considered as part of this invention.

In one embodiment, this invention provides an implantable scaffolding material, which may have varying mechanical properties to fit the application as to the desired implantation site of the scaffold.

In some embodiments, the materials/matrices of this invention are moldable, in that they may be so delivered and applied as to fill voids, and conform in shape, to fit a region of application.

In addition, according to the concepts of the present disclosure, the material/matrix can be produced so as to not only have the appropriate physical microstructure to enable desired cellular activity upon implantation, but also the biochemistry (collagens, growth factors, glycosaminoglycans, etc.) naturally found in tissues where the material/matrix is implanted for applications such as, for example, tissue repair or regneration, cosmetic skin filler, controlled release of an agent, or other applications, as will be appreciated by one skilled in the art.

In one embodiment, the methods/materials of the invention are used for wound healing.

In one embodiment, the term “wound” refers to damaged biological tissue. In one embodiment, the wound is a laceration of the skin. In one embodiment, the wound may be an abrasion of the skin with two separated parts of tissue which in another embodiment, need to be brought together. In one embodiment, the wound may refer to a surgical incision. In another embodiment, the wound may involve damage to lung tissue, arterial walls, or other organs with elastic fibers. In one embodiment, the wound may involve an abscess, or in another embodiment, the wound may be exacerbated by diabetes. In one embodiment, the methods and materials of the invention are used to accelerate wound healing.

According to this aspect of the invention and in one embodiment, wound healing may comprise fibrin clot formation, recruitment of inflammatory cells, reepitheliazation, and matrix formation and remodeling and as such, the materials/matrices of this invention in one embodiment or the methods of use in another, may incorporate molecules involved in these stages within the material/matrix. In one embodiment, stimulation of wound healing may be accomplished using encapsulated thrombin, as described herein.

In another embodiment, the materials/matrices of this invention are seeded with epidermal cells or mesenchymal stem cells, and one or more extracellular matrix components or analogs thereof, used to heal an open wound, or promote cartilage or bone formation, by injecting the material/matrix into the wound, or desired site for bone/cartilage formation, respectively. In some embodiments, the materials/matrices comprise elastin, hyaluronic acid, fibronectin, neutrophils, monocytes and EGF, osteoblasts, chondrocytes, and bone morphogenetic proteins, etc. In another embodiment, the material/matrix is additionally seeded with stem cells, which in one embodiment are engineered to express relevant factors.

In some embodiments, the materials/matrices and/or methods of this invention are for the treatment, repair or regeneration of the skin. In some embodiments, according to this aspect, such treatment, repair or regeneration is with respect to a skin wrinkle or lesion, to address a disease, disorder, condition or normal aging, or sun damage of the skin.

In some embodiments, the injectable materials/matrices of this invention may serve as a filler for a skin wrinkle or lesion in the subject, or in some embodiments, as a cosmetic overlay.

In some embodiments, this invention provides a method of controlled delivery of an agent in a subject, the method comprising administering a controlled delivery material to a subject, the material comprising:
∘ a polymer solution;
∘ a thermo-responsive liposome dispersed in said solution;
∘ a zero-length cross-linking agent encapsulated in said liposome; and
∘ at least one agent of interest encapsulated in said liposome;
   wherein the polymer and the cross-linking agent are segregated at a first temperature, non-segregated at a second temperature, and the controlled delivery material is injectable at the first temperature.

In some embodiments, the term controlled delivery of an agent of interest refers to its ordinary meaning in the art. In some embodiments, the term “controlled delivery” refers to the amount, duration and/or timing of the release of the agent. Such release kinetics may be a function of its contact with a material or compound dispersed in the polymer solution, or in some embodiments, activation as a result of contact with a material or compound dispersed in the polymer solution, or in some embodiments, diffusion from the encapsulated liposomes to a target site, or combinations thereof. In some embodiments, the term controlled release may also refer to a release profile of the agent customized for a particular subject or groups of subjects, as a function of release kinetics in the subject. Other agents may be incorporated in the materials of this invention to hasten or delay release, or alter the quantity of agent released, or combinations thereof.

In some embodiments, the controlled release of the agent of interest via the materials/matrices of this invention is by osmotic release. For example, in some embodiments, the solution is free of swellable polymers and comprises nonswelling solubilizing agents and wicking agents, and the encapsulated agent is admixed with the polymer following achievement of the optimal temperature. In some embodiments, the non-swelling solubilizing agent enhances the solubility of the agent, and thereby its release.

In some embodiments, the controlled release formulation and thereby kinetics of release can be designed to mimic a desired plasma profile, and comprises an embodiment of this invention.

In some embodiments, the controlled delivery material comprises two or more agents.

In some embodiments, a second agent of interest is dispersed in the polymer solution, which in some embodiments is a precursor molecule, and said at least one agent processes said precusor to form a final product for delivery to said subject. In some embodiments, such precursor may comprise a protein, which undergoes proteolytic processing for conversion to a product which performs an effector function, or in another embodiment, undergoes glycosylation, or other processing to produce an active compound. In some embodiments, such precursor may be encapsulated or dispersed in the polymer solution, with a molecule, which is capable of providing appropriate processing being in polymer solution, or encapsulated, respectively, such that processing can occur only upon achievement of the desired temperature for activity. For example, proteins expressed in prokaryotes, which will be active in human subjects upon processing are encapsulated in the liposomes, and N-terminal methionyl removal from such bacterially expressed proteins is accomplished, once in contact with cathepsin C, also referred to as dipeptidyl-aminopeptidase 1 (DAP-1), which may be suspended or solubilized in the polymer solution.

It is to be understood that any application, wherein the materials/matrices and methods utilizing the same may be useful are envisioned as part of this invention, representing an embodiment thereof.

The following examples are presented in order to more fully illustrate the preferred embodiments of the invention. They should in no way be construed, however, as limiting the broad scope of the invention

### EXAMPLES

### Materials and Methods

### Liposomes Synthesis

Liposomes were synthesized with varying concentrations of dipalmitoyl-phosphatidylcholine (DPPC), dimyristoylphosphatidylcholine (DMPC), and cholesterol. HRP was encapsulated by the dehydration-rehydration vesicle (DRV) method [C. Kirby and G. Gregoriadis, Bio-Technology 2, 979 (1984)]. The lipid-cholesterol mixture was dissolved in chloroform, which was then evaporated to form a film in a round-bottom flask. The film was incubated with deionized water (60 mg of lipid/mL) at 50° C. for 30 min with intermittent vortexing. The solution was sonicated with a probe-tip sonicator at 55° C. until optically transparent. After the solution had cooled to room temperature, an equal volume of enzyme solution (2 mg/mL of HRP in water) was added. The mixture was then frozen and freeze-dried for at least 24 h. The liposomes were rehydrated with phosphate buffer solution (PBS), and incubated at room temperature for 30 min. Unencapsulated HRP was then rinsed off by repeated centrifugation (2,000 g, 1 min) with water, PBS, sodium chloride, calcium chloride, or magnesium chloride solution. The lipids and cholesterol were purchased from Avanti Polar Lipids and Alfa Aesar, respectively. All other chemicals were purchased from Sigma-Aldrich.

### HRP Release Studies

HRP release was measured by adding PBS to a given volume of liposomes. The mixture was then incubated for a set amount of time either at room temperature or 37° C. The liposomes were then centrifuged into a pellet, and the HRP concentration in the supernatant was measured calorimetrically with the Amplex Red dye (Molecular Probes) at 560 nm. Release was measured in terms of units of enzymatic activity derived from each mL of liposomes, where there are approximately 300 mg of phospholipid per mL of liposome.

### Hyaluronic Acid-Tyramine Conjugation

HA-Tyr conjugation was carried out as described by Kurisawa et al. [Chemical Communications 34, 4312 (2005)]. Briefly, tyramine was covalently attached to the hyaluronic acid (90-150 kDa) backbone by a reaction with N-(3-dimethylaminopropyl)-N′-ethylcarbodiimide (EDC) and N-hydroxysuccinimide (NHS). Substitution of tyramine was verified by nuclear magnetic resonance (NMR) spectroscopy, and ranged from 3% to 7%. Unreacted tyramine and the by-products of the reaction were removed by dialysis. Hyaluronic acid and EDC were purchased from Lifecore and Alfa Aesar, respectively. Tyramine and NHS were purchased from Sigma-Aldrich.

### Gelation Kinetic Studies

Hydrogels were formed by adding HRP liposomes to HA-Tyr (25 mg/1.5 mL PBS)/H₂O₂ (5 wt %, 5 μL per 25 mg of HA-Tyr) solutions. The gelation of the hydrogel networks was studied by oscillatory rheometry on a TA Instrument AR 2000. HA-Tyr/H₂O₂/HRP liposome solutions were pipetted onto a temperature-controlled base plate (set at 20° C. or 37° C.) upon which a 60-mm, 2° cone was lowered. Measurements were taken at a controlled strain of 1% and at a frequency of 1.0 Hz. The point at which the storage modulus (G′) and the loss modulus (G″) intersected was considered the gel point.

### In Situ Gelation

Aseptically prepared HRP liposomes were combined with sterile-filtered HA-Tyr/H₂O₂ solution (composition described above). The mixture was then injected subcutaneously into the dorsum of a BALB/c mouse. The mouse was sacrificed after ˜2 h, and the gel that had formed was harvested and analyzed.

### Implantation of Cell-Seeded Scaffold

Chondrocytes were isolated from the cartilage of the femur, patella and tibia of porcine legs (5-6 months old) that were obtained from the local abattoir. The tissue was cut into small pieces (10 mm³), and digested in a Type II collagenase solution (0.2 wt % in F-12 medium supplemented with 0.1 M non-essential amino acids, 0.5 μg/mL of fungizone, 1 wt % penicillin-streptomycin, and 0.4 mM L-proline) at 37° C. for ˜12 h. The cell solution was then filtered through a 100-μm strainer and centrifuged (8000 g, 10 min) twice with PBS. The collagenase was purchased from Gibco. The F-12 medium, non-essential amino acids and fungizone were purchased from Invitrogen. The penicillin-streptomycin and L-proline were purchased from Sigma.

Cells were then suspended in sterile-filtered HA-Tyr (13 mg per 1.5 mL of F-12 medium supplemented with 0.1 M non-essential amino acids, 0.5 μg/mL of fungizone, 1 wt % penicillin-streptomycin, 0.4 mM L-proline and 10 wt % fetal bovine serum)/H₂O₂ (5 wt %, 5 μL per 25 mg of HA-Tyr) solution at a concentration of 50×10⁶/mL. 50 μL of the cell solution was combined with 5 μL of HRP (0.125 mg/mL), and placed in a mold to create a cylindrical gel with a thickness of ˜1 mm. The gels were then implanted subcutaneously in the dorsum of Scid mice (n=4), which were sacrificed at Day 14 and 28. The constructs were harvested, and analyzed by histology with the Safranin ◯ and Masson's trichrome stains. Safranin ◯ was purchased from Sigma, and the Masson's trichrome kit was purchased from Richard-Allen Scientific.

### Example 1

### Thermally Triggered Cross-linker Release from Liposomes as a Basis for Scaffold Design

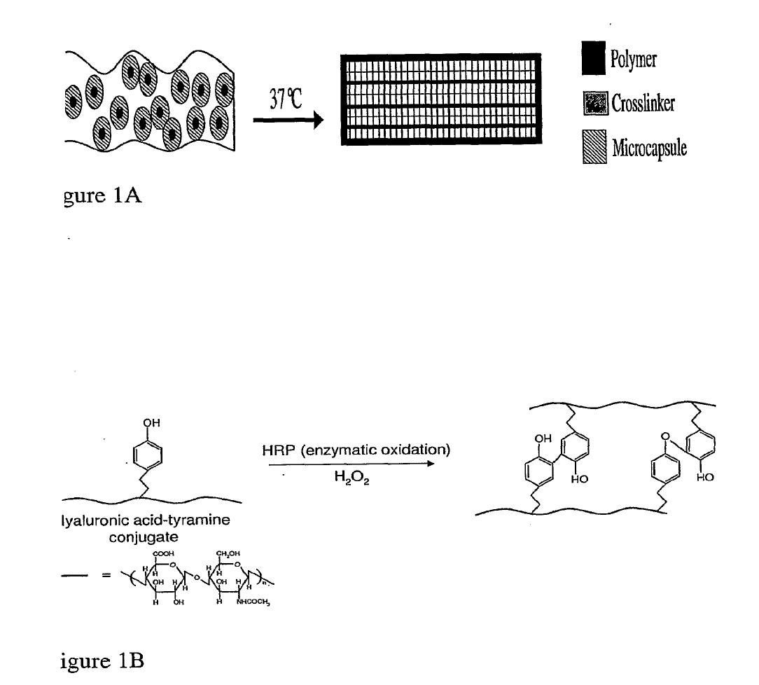

In order to provide a biocompatible, injectable scaffolding system, the encapsulation of a cross-linker, or active species, was conducted within thermoresponsive liposomes (FIG. 1). At room temperature, the construct remains a liquid due to the separation of the polymer and cross-linker. At body temperature, the cross-linker is released, resulting in solidification and scaffold formation. Liposomes are biocompatible, versatile encapsulating agents that have been studied extensively for drug and gene delivery. They are also known to experience a gel-to-liquid phase transition at a given temperature, where the membrane permeability is significantly enhanced. We have, therefore, chosen liposomes for the thermally triggered release of crosslinkers. By segregating the crosslinker from the polymer, determinants of working time and mechanical strength can be separately controlled. The working time depends only on temperature, while mechanical strength depends on the amount of crosslinker encapsulated. This method allows the clinician plenty of time to inject the scaffold at room temperature, without sacrificing the mechanical strength of the final construct.

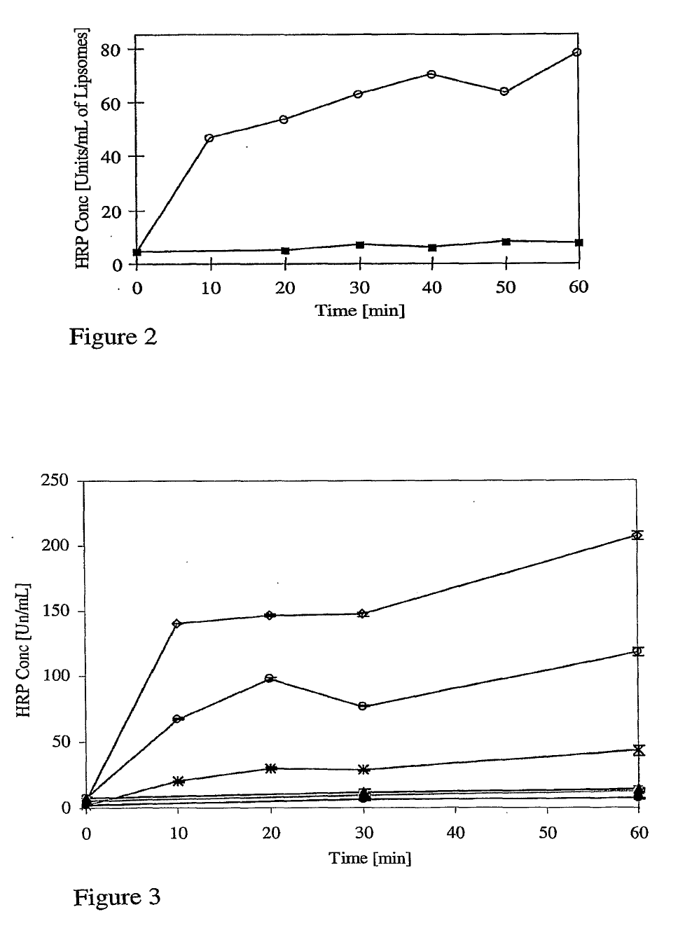

The method was applied to create a thermally triggered hyaluronic acid scaffold. Hyaluronic acid is a naturally occurring polysaccharide that is biocompatible, nonantigenic, bioresorbable, and one of the chief components of the extracellular matrix (ECM). It has been used extensively in applications, ranging from synovial joint fluid supplementation to cosmetic wrinkle fillers, and shows great promise as a tissue engineering scaffold. As a model system, a hyaluronic acid-tyramine conjugate (HA-Tyr) system that can be crosslinked under physiological conditions in the presence of horseradish peroxidase (HRP) was chosen (FIG. 2). HRP isa zero-length cross-linker that enzymatically oxidizes and activates the tyramine groups, which then react with each other to form a hydrogel network.

In order to impart temperature sensitivity to this system, HRP was encapsulated within thermoresponsive liposomes, which were then dispersed in an HA-Tyr/H₂O₂ solution. This hyaluronic acid solution was able to remain a liquid at room temperature for several hours, while gelling to form a scaffold at body temperature within minutes.

### Example 2

### Effect of Material Parameters on Total HRP Release

To minimize enzyme deactivation and avoid the heat and organic solvents involved in general liposome synthesis procedures, a DRV method, as described, was chosen to encapsulate HRP. This method involved the synthesis of blank single unilamellar vesicles (SUV's), with which an HRP solution was mixed. The liposome/enzyme solution was then freeze-dried, during which the vesicles fused and thus encapsulated the enzymes. Upon rehydration, liposomes containing HRP within the aqueous core were then formed. Following this technique, HRP remained encapsulated within the liposome at 25° C., and was released rapidly at 37° C. (FIG. 2).

In order to create liposomes which may be thermally triggered at body temperature, dipalmitoyl-phosphatidylcholine (DPPC) and dimyristoylphosphatidylcholine (DMPC) were used. These phospholipids have phase transition temperatures of 41° C. and 23° C., respectively, and may be blended to give an overall phase transition temperature of 37° C. Varying the DPPC/DMPC weight ratio was found to significantly influence the release profile of the enzyme (FIG. 3). As the fraction of DPPC increased, the overall phase transition temperature increased, thereby decreasing permeability at 37° C.

Cholesterol was added to modify liposomal bilayer fluidity and decrease enzyme leakage at room temperature. The improvement to room-temperature stability was small, and HRP encapsulation and liposome size was reduced (FIG. 4). Cholesterol, in some instances rigidifies/stabilizes liposome membranes, but at a potential hindrance in some instances of the fusion of liposomes during the dehydration process. This process of liposome fusion led to HRP encapsulation.

### Example 3

### Effect of Processing Parameters on Total HRP Release

Due to the sensitivity of the enzyme to deactivation, the processing parameters were found to be critical in determining total HRP release. Increased lipid concentration increased HRP entrapment (FIG. 5A), since the contact between the EHP and lipid vesicles increased. During dehydration, an increase in lipid concentration increased the probability of contact between HRP molecules and lipid sheets, thereby increasing total encapsulation during vesicle fusion.

Freezing stresses can lead to significant denaturation of the protein during liposome synthesis. FIG. 5B shows that the means by which the enzymelliposome solutions are frozen, which determines the rate of cooling, is important in determining the enzymatic activity recovered. The slower the freezing rate, the less the enzyme is damaged in the course of synthesis.

In freeze-drying proteins alone, it has been found that the addition of a cryoprotectant, such as trehalose, aids in preserving enzymatic activity. However, any such beneficial effects were negated by the disruption to liposome formation, which consequently resulted in lower encapsulation (FIG. 6).

It was also found that varying the volume of solution used for rehydration greatly affects the final HRP concentration observed (FIG. 7). During the rehydration process, the lipid blanketed the surrounding solution to form a liposome. As the volume of solution used for rehydration increased, the HRP was diluted, thereby decreasing the HRP concentration within each liposome.

In addition, sonicating the liposomes prior to freezing was observed to increase the encapsulation of the enzyme (FIG. 8). The sonication step breaks up the liposomes, and converts the multi-lamellar vesicles (MLV's) with an onion-like structure to single unilamellar vesicles (SWV's).

Sonication broke up the liposome agglomerations and dispersed the vesicles homogeneously within the solution, increasing the interaction between the lipid and HRP, and thereby improving encapsulation.

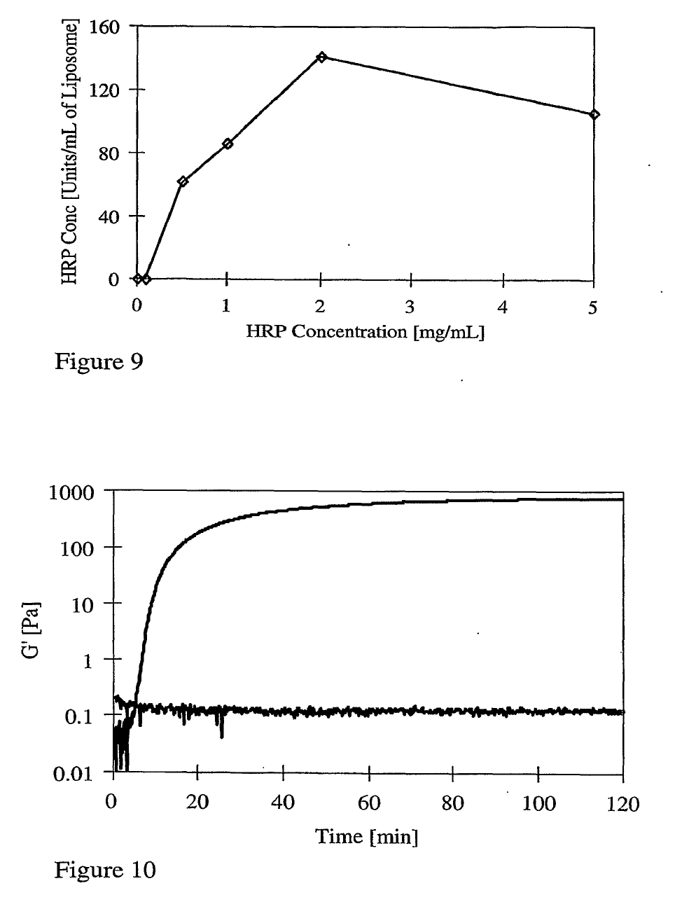

The HRP release at 37° C. can also be tuned by the initial HRP concentration of the solution added to the empty liposomes prior to freezing. FIG. 9 shows that as the HRP concentration increased, the HRP encapsulated increased. However, above an HRP concentration of 2 mg/mL, the protein (which also has surfactant properties) would stabilize the liposomes during dehydration and inhibit liposome fusion, causing decreased encapsulation.

### Example 4

### Modulation of Gelation Time

When ERP liposomes were suspended in the HA-Tyr/H₂O₂ solution and heated to 37° C., the elastic modulus, G′, began to rise within 3 minutes, indicating the onset of gelation (FIG. 10). The gel point was reached in ˜7 min. On the other hand, at 20° C., the solution remained liquid for several hours, and the magnitude of G′ remained small and constant over time. This behavior and time frame reflected the HRP release observed in FIG. 2.

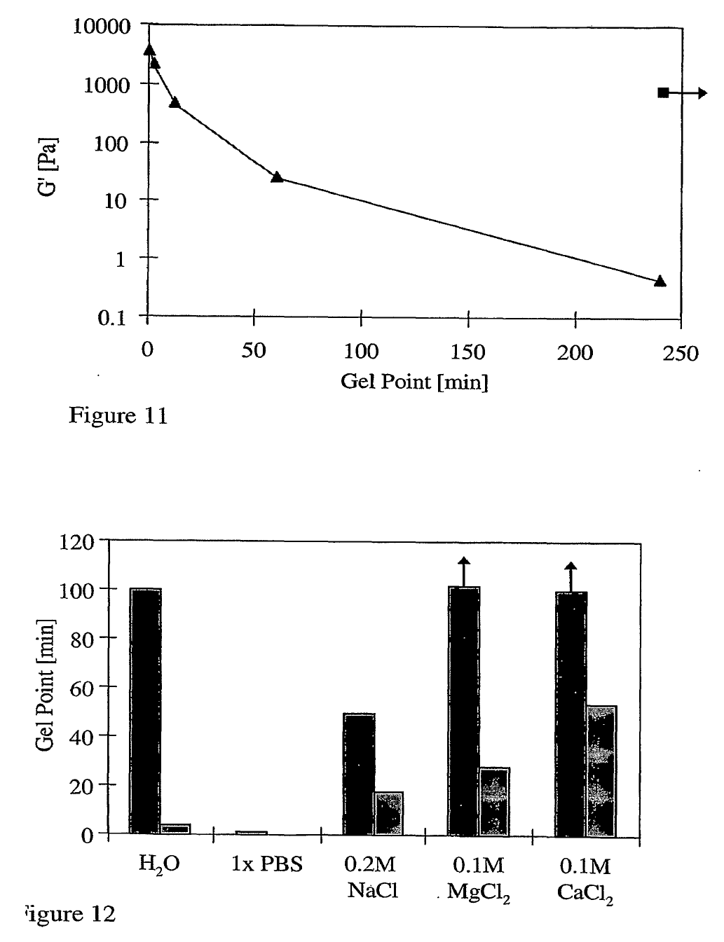

The benefit of the HRP Hiposome system is illustrated in FIG. 11. The working time and mechanical strength of polymer-crosslinker systems were previously generally modulated by adjustments to crosslinker concentration. For the HA-Tyr system, the working time was gauged by the gel point, while mechanical strength was measured by the storage modulus, G′. The inverse relationship between G′ and the gel point of the HA-Tyr/H₂O₂/HRP system is illustrated in FIG. 11 by varying the HRP concentration; it was shown that there was a trade-off between the working time and mechanical strength. By utilizing thermally triggered liposomes to encapsulate HRP, though, this was no longer the case. A working time of several hours was achieved at room temperature without sacrificing a high G′ value.

For various clinical needs, gelation times (at both 20° C. and 37° C.) could be tuned by adjusting the total HRP released, which can be readily controlled by the parameters described above. In addition, the liposome, H₂O₂ and polymer concentrations could be optimized to arrive at the desired gelation kinetics.

In order to maximize the gelation time at room temperature without compromising the gelation time at 37° C., it was found that the solution used to rinse off the liposomes after rehydration was critical. It is known that proteins tend to stick to and destabilize the liposome membrane. FIG. 12 shows that removal was dependent on the composition of the wash solution used. PBS and NaCl solutions activated the HRP bound to the surface of the liposomes, and caused rapid gelation at room temperature. Conversely, divalent cations may deactivate and cause unfolding of the HRP.

### Example 5

### Cartilage Formation In Vivo Via the Scaffolds of this Invention

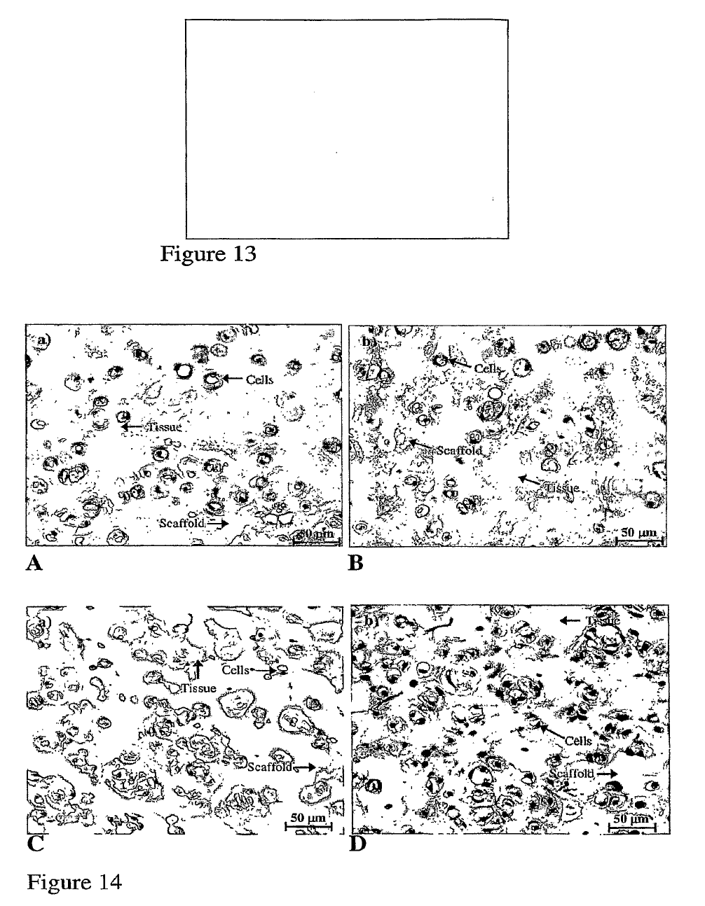

Preliminary in vivo studies were conducted by injecting an HA-Tyr/H₂O₂/HRP liposome solution subcutaneously applied to a mouse model. The solution remained a liquid at room temperature and was injected without difficulty through a syringe. Based on palpitation, the solution cured within 5 minutes of exposure to body temperature, and an intact gel was harvested upon sacrifice of the mouse (FIG. 13). Therefore, it did not appear that there were interfering reactions in vivo that inhibited the enzyme release or crosslinking reaction.

In order to study the potential of the HA-Tyr polymer as a scaffold material, cartilage cells, harvested from porcine joints, were encapsulated within HA-Tyr/H₂O₂/HRP gels. The cell-seeded scaffolds were then implanted subcutaneously into a mouse model. After harvesting the tissue, the constructs were stained for glycosamninoglycans (Safranin ∘) (FIGS. 14 A & B) and collagen (Masson's trichrome) (FIGS. 14C & D), both of which are major components of cartilage tissue. The cells survived the encapsulation process, and retained the phenotype typical of chondrocytes, with a rounded shape, small nucleus and large cytoplasm. Within two weeks, the chondrocytes showed prominent rings of tissue, containing glycosarminoglycan and collagen, deposited onto the surrounding scaffold. At Day 28, the areas of tissue were shown to increase and progressively replace the scaffold material.

### Example 6

### Thrombin Release from Thermoresponsive Liposomes

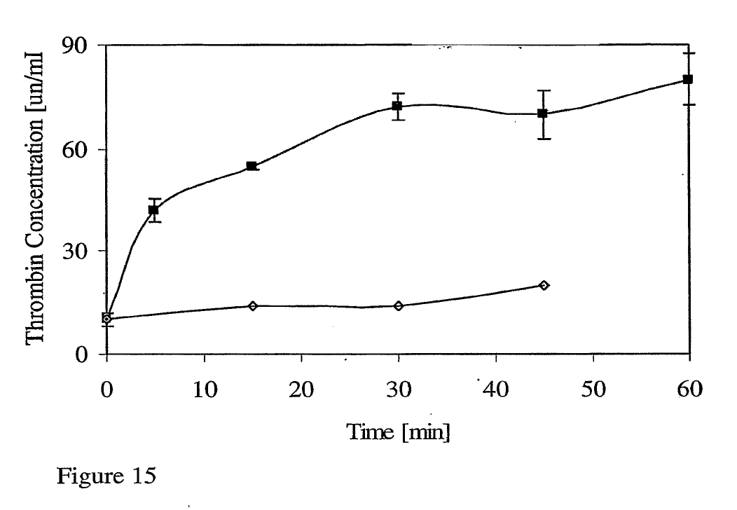

Thrombin is a critical enzyme in the coagulation cascade of blood. It activates the monomer, fibrinogen, which then assembles into a fibrin network. In order to determine whether a fibrinogen polymerization could be accomplished, thrombin was encapsulated in lipsomes, as described herein, by the dehydration-rehydration method. Total release was measured by incubating a set volume of thrombin liposomes with a set volume of PBS at either 25° C. or 37° C. After a given amount of time, the liposomes were centrifuged at 20,000 g for 10 minutes, and the concentration of thrombin in the supernatant was measured susing Coomassie dye. FIG. 15 demonstrates that at 37° C., thrombin was rapidly released from the liposomes, while at 25° C. thrombin remained essentially encapsulated.

It is proposed that the thrombin liposomes can be dispersed in a fibrinogen solution. At room temperature, the precursor solution remains a liquid because the monomer and activating agent are segregated. At body temperature, thrombin is released and the solution solidifies into a fibrin network.

This material can, in some embodiments, be used as a thermoresponsive injectable scaffold for tissue engineering or an injectable therapy for hemophiliacs, whose natural clotting ability is impaired.

A thermally triggered injectable scaffold has been created by utilizing thermoresponsive liposomes to segregate a crosslinker or active species from a polymer. The precursor solution remained as a liquid until it was exposed to body temperature, whereby the crosslinker was released for the scaffold formation. The crosslinker release and gelation kinetics, in some embodiments, are tailored for a given application by tuning various material and processing parameters. HA-Tyr and HRP liposomes were used as a model system in this study to demonstrate the feasibility of this approach. The HA-Tyr/H₂O₂/HRP liposome solution was stable as a liquid at room temperature for several hours, but would solidify upon exposure to body temperature. Preliminary in vivo studies also showed the regenerative potential of this system as a tissue engineering scaffold.

This technology could potentially be extended to impart temperature sensitivity to various polymer-crosslinker systems that previously required implantation. In some embodiments, the thermally triggered system could also be useful as an injectable cosmetic soft tissue filler or drug delivery vehicle.

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. An injectable scaffolding material, comprising:
a. a polymer solution;
b. a thermo-responsive liposome dispersed in said solution; and
c. a zero-length cross-linking agent encapsulated in said liposome;
wherein said polymer and said cross-linking agent are segregated at a first temperature, non-segregated at a second temperature, and said scaffolding material is injectable at said first temperature.

2. The injectable scaffolding material of claim 1, wherein said polymer solution comprises poly (pyranose), poly(hydroxyl acid), poly(lactone), poly (amino acid), poly(anhydride), poly (orthoester), poly (phosphazine), poly(ethylene glycol) or poly(pbosphoester).

3. The injectable scaffolding material of claim 1, wherein said polymer solution comprises a collagen, a glycosaminoglycan, or a combination thereof.

4. The injectable scaffolding material of claim 1, wherein said thermo-responsive liposome comprises a mixture of lipids, phospholipids, or a combination thereof.

5. The injectable scaffolding material of claim 4, wherein said a lipid is cholesterol.

6. The injectable scaffolding material of claim 4, wherein said a phospholipid is a dipalmitoyl-phosphatidylcholine (DPPC), dimyristoylphosphatidylcholine (DMPC), distearoyl-phosphatidylcholine or a combination thereof.

7. The injectable scaffolding material of claim 4, wherein said liposome comprises two or more phospholipids.

8. The injectable scaffolding material of claim 7, wherein the relative concentration of one of said two or more phospholipids is varied, so as to increase permeability of said liposome.

9. The injectable scaffolding material of claim 8, further comprising a lipid, which inreases fluidity of said liposome.

10. The injectable scaffolding material of claim 4, wherein said first temperature is less than the phase transition temperature of said mixture.

11. The injectable scaffolding material of claim l, wherein said second temperature is about 37° C.

12. The injectable scaffolding material of claim 1, wherein said zero-length cross-linking agent is (1 ethyl 3-(3dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or a combination thereof.

13. The injectable scaffolding material of claim 1, wherein said zero-length cross-linking agent is an enzyme.

14. The injectable scaffolding material of claim 1, wherein said enzyme is a transglutaminase, peroxidase, an oxidase, or a combination thereof.

15. The injectable scaffolding material of claim 1, wherein the concentration of said polymer ranges from 0.1-50% w/w of said material.

16. The injectable scaffolding material of claim 1, wherein the concentration of said polymer ranges from 40-95% w/w of said material

17. The injectable scaffolding material of claim 1, further comprising a compound or cell of interest.

18. A cosmetic filler, drug delivery vehicle or scaffold for tissue engineering or repair, comprising the injectable scaffolding material of claim 1.

19. A process for preparing an injectable scaffolding material, comprising the steps of:
a. preparing a mixture of lipids, phopsholipids, or a combination thereof and a cross-linking agent;
b. freezeing said mixture;
c rehydrating said mixture to form liposomes encapsulating said cross-linking agent; and
d. dispersing said liposomes in a liquid comprising a polymer;
wherein said dispersing is conducted at a temperature which is less than the phase transition temperature of said mixture and said cross-linking agent or reactive species and said polymer are segregated at said temperature.

20. The method of claim 19, wherein said freezing is accomplished at a rate of between 0.5-100° C. per minute.

21. The method of claim 19, further comprising the step of sonicating said mixture prior to freezing, in step (b).

22. The method of claim 19, wherein said rehydrating is conducted with a salt solution with a monovalent or divalent cation.

23. The method of claim 22, wherein said salt solution comprises sodium chloride.

24 The method of claim 19, wherein said polymer comprises poly (pyranose), poly(hydroxyl acid), poly(lactone), poly (amino acid), poly(anhydride), poly (orthoester), poly (phosphazine), poly(ethylene glycol) or poly(phosphoester).

25. The method of claim 19, wherein said polymer comprises a collagen, a glycosaminoglycan, or a combination thereof.

26. The method of claim 19, wherein said a lipid is cholesterol.

27. The method of claim 19, wherein said phospholipid is a dipalmitoyl-phosphatidylcholine (DPPC), dimyristoylphosphatidylcholine (DMPC), distearoyl-phosphatidylcholine or a combination thereof.

28. The method of claim 19, wherein said mixture comprises two or more phospholipids.

29. The method of claim 28, wherein the relative concentration of one of said two or more phospholipids is varied, so as to increase permeability of said liposome.

30. The method of claim 28, wherein said mixture further comprises a lipid, which inreases fluidity of said liposome.

31. The method of claim 28, wherein said lipid is at a concentration of from about 5 to about 70 milligrams per milliliter.

32. The method of claim 19, wherein said method is conducted at a temperature, which is less than the phase transition temperature of said mixture.

33. The method of claim 19, wherein said zero-length cross-linking agent is (1 ethyl 3-(3dimethyl aminopropyl)carbodiimide (EDAC), N-Sulfohydroxy succinamide (Sulfo NHS), 5-iodopyrimidines, N-carbaLkoxydihydroquinolines, pyrroloquinolinequinones, or a combination thereof.

34. The method of claim 19, wherein said zero-length cross-linking agent is an enzyme.

35. The method of claim 34, wherein said enzyme is a transglutaminase, peroxidase, xanthine oxidase, or a combination thereof.

36. The method of claim 19, wherein the concentration of said polymer ranges from 1-85% w/w of said material.

37. The method of claim 19, wherein said liquid further comprises a cell, a compound of interest, or a combination thereof.

38. The method of claim 37, wherein said compound of interest is a nucleic acid, a hormone, a growth factor, a cytokine, a chemokine, a peptide, a drug or a combination thereof.

39. The method of claim 19, wherein said rehydrating is accomplished in a small volume.

40. The method of claim 39, wherein said volume ranges from about 1/10 to about 1/1 of the volume for said preparing in (a).

41. An injectable scaffolding material prepared according to the process of claim 19.

42. A method of tissue repair, regeneration, or organogenesis in a subject, the method comprising:
a. administering a scaffolding material to a subject, said scaffolding material comprising:
i. a polymer solution;
ii. a thermo-responsive liposome dispersed in said solution; and
iii. a zero-length cross-linking agent encapsulated in said liposome;
wherein said polymer and said cross-lnkilng agent are segregated at a first temperature, non-segregated at a second temperature, and said scaffolding material is injectable at said first temperature.

43. The method of claim 42, wherein said scaffolding material further comprises cells involved in tissue repair, regeneration, or organogenesis.

44. The method of claim 42, wherein said scaffolding material further comprises a tissue promoting factor.

45. The method of claim 42, wherein said tissue promoting factor is a hormone, a nucleic acid, a growth factor, a chemokine, a peptide, an enzyme or a combination thereof.

46. The method of claim 42, wherein said polymer comprises poly (pyranose), poly(hydroxyl acid), poly(lactone), poly (amino acid), poly(anhydride), poly (orthoester), poly (phosphazine), poly(ethylene glycol) or poly(phosphoester).

47. The method of claim 42, wherein said polymer comprises a collagen, a glycosaminoglycan, or a combination thereof.

48. The method of claim 42, wherein said a lipid is cholesterol.

49. The method of claim 42, wherein said phospholipid is a dipalmitoyl-phosphatidylcholine (DPPC), dimyristoylphosphatidylcholine (DMPC), distearoyl-phosphatidylcholine or a combination thereof.

50. The method of claim 42, wherein said mixture comprises two or more phospholipids.

51. The method of claim 50, wherein the relative concentration of one of said two or more phospholipids is varied, so as to increase permeability of said liposome.

52. The method of claim 50, wherein said mixture further comprises a lipid, which inreases fluidity of said liposome.

53. The method of claim 42, wherein said scaffolding material is held at a temperature, which is less than the phase transition temperature of said mixture, prior to said administering.

54. The method of claim 42, wherein said zero-length cross-linking agent is (1 ethyl 3-(3dimethyl aminopropyl)carbodiimide (EDAC), N-Sulfohydroxy succinamide (Sulfo NIHS), 5-iodopyrimidines, N-carbalkoxydihydroquinolines, pyrroloquinolinequinones, or a combination thereof.

55. The method of claim 42, wherein said zero-length cross-linking agent is an enzyme.

56. The method of claim 55, wherein said enzyme is a transglutaminase, peroxidase, xanthine oxidase, or a combination thereof.

57. The method of claim 42, wherein the concentration of said polymer ranges from 1-85% w/w of said material.

58. The method of claim 42, wherein said tissue repair or regeneration is of the skin.

59. The method of claim 58, wherein said repair or regeneration is of a skin wrinkle or lesion.

60. The method of claim 58, wherein said injectable scaffolding material serves as a filler for a skin wrinkle or lesion in said subject.

61. A method of controlled delivery of an agent in a subject, the method comprising:
a. administering a controlled delivery material to a subject, said material comprising:
i. a polymer solution;
ii. a thermo-responsive liposome dispersed in said solution;
iii. a zero-length cross-linking agent encapsulated in said liposome; and
iv. at least one agent of interest encapsulated in said liposome;
wherein said polymer and said cross-linking agent are segregated at a first temperature, non-segregated at a second temperature, and said controlled delivery material is injectable at said first temperature.

62. The method of claim 61, wherein said controlled delivery material comprises two or more agents.

63. The method of claim 61, wherein said a second agent of interest is dispersed in said polymer solution.

64. The method of claim 63, wherein said second agent is. a precursor molecule, and said at least one agent processes said precusor to form a final product for delivery to said subject.

65. The method of claim 61, wherein said polymer comprises poly (pyranose), poly(hydroxyl acid), poly(lactone), poly (amino acid), poly(anhydride), poly (orthoester), poly (phosphazine), poly(ethylene glycol) or poly(phosphoester).

66. The method of claim 61, wherein said polymer comprises a collagen, a glycosaminoglycan, or a combination thereof.

67. The method of claim 61, wherein said a lipid is cholesterol.

68. The method of claim 61, wherein said phospholipid is a dipalmitoyl-phosphatidylcholine (DPPC), dimyristoylphosphatidylcholine (DMPC), distearoyl-phosphatidylcholine or a combination thereof.

69. The method of claim 61, wherein said mixture comprises two or more phospholipids.

70. The method of claim 69, wherein the relative concentration of one of said two or more phospholipids is varied, so as to increase permeability of said liposome.

71. The method of claim 69, wherein said mixture further comprises a lipid, which inreases fluidity of said liposome.

72. The method of claim 61, wherein said controlled delivery material is held at a temperature, which is less than the phase transition temperature of said mixture, prior to said administering.

73. The method of claim 61, wherein said zero-length cross-linking agent is (1 ethyl 3-(3dimethyl aminopropyl)carbodiimide (EDAC), N-Sulfohydroxy succinamide (Sulfo NHS), 5-iodopyrimidines, N-carbalkoxydihydroquinolines, pyrroloquinolinequinones, or a combination thereof.

74. The method of claim 61, wherein said zero-length cross-linking agent is an enzyme.

75. The method of claim 74, wherein said enzyme is a transglutaminase, peroxidase, xanthine oxidase, or a combination thereof.

76. The method of claim 61, wherein the concentration of said polymer ranges from 1-85% w/w of said material.
